(19) United States
(12) Patent Application Publication
(10) Pub. No.: US 2022/0411471 A1
(43) Pub. Date: Dec. 29, 2022

(54) CELL-PERMEABLE CYCLIC PEPTIDES AND USES THEREOF

(71) Applicant: Unnatural Products Inc., Santa Cruz, CA (US)

(72) Inventors: Cameron PYE, Santa Cruz, CA (US); Joshua SCHWOCHERT, Santa Cruz, CA (US); Seth RUBIN, Santa Cruz, CA (US); Anthony SILVESTRI, Santa Cruz, CA (US)

(21) Appl. No.: 17/750,110

(22) Filed: May 20, 2022

Related U.S. Application Data
(63) Continuation of application No. PCT/US2020/061596, filed on Nov. 20, 2020.
(60) Provisional application No. 63/047,178, filed on Jul. 1, 2020.
(60) Provisional application No. 62/938,864, filed on Nov. 21, 2019.

(51) Int. Cl. C07K 7/64; A61P 35/02

(52) U.S. Cl. CPC C07K 7/64; A61P 35/02

(57) ABSTRACT

Cyclic peptides that inhibit MDM2 or MDM2 and MDM4, pharmaceutical compositions containing these cyclic peptides, and methods of using these cyclic peptides for inhibiting MDM2 or MDM2 and MDM4 are described herein.

### CROSS-REFERENCE

This application is a continuation of International Application No. PCT/US2020/061596, filed Nov. 20, 2020, which claims priority to U.S. Provisional Application No. 62/938,864, filed Nov. 21, 2019, and U.S. Provisional Application No. 63/047,178, filed Jul. 1, 2020, each of which is incorporated by reference herein in its entirety for all purposes.

### SUMMARY

Disclosed herein, in certain embodiments, are compounds, pharmaceutical compositions comprising the compounds, and the use of the compounds in the treatment of disease. Further, the disclosure relates to cyclic peptides useful as MDM2 or dual MDM2/MDM4 inhibitors, compositions and uses thereof in the treatment of diseases such as cancer. Additionally, the disclosure relates to cyclic peptides useful as MDM2 or dual MDM2/MDM4 inhibitors, compositions and uses thereof to induce the death of a senescent cell, and particularly to treat a disease or disorder associated with the proliferation of senescent cells.

In one aspect, the present disclosure provides a cyclic peptide comprising:

nine to eleven amino acid residues independently selected from amino acid residues that are not charged at physiological pH; P a first and a second beta hairpin region; and characterized by one of the following:

at least four amino acid residues comprising rings independently selected from optionally substituted monocyclic carbocycle and optionally substituted monocyclic heterocycle, wherein at least one of the monocyclic carbocycle and monocyclic heterocycle are substituted;

at least four amino acid residues with side chains selected from -alkylene-(monocyclic carbocycle) and -alkylene-(monocyclic heterocycle), wherein the monocyclic carbocycle and monocyclic heterocycle are independently optionally substituted; and

at least three amino acid residues comprising rings independently selected from optionally substituted phenyl and optionally substituted monocyclic heteroaryl.

In some embodiments, the first beta hairpin region comprises two contiguous amino acid residues. In some embodiments, the first beta hairpin region comprises two contiguous residues independently selected from: L-Pro, D-Pro, L-Aze, D-Pip, L-NMe-Phe, and D-NMe-Val, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the first beta hairpin region comprises two contiguous residues independently selected from: L-Pro, D-Pro, L-Aze, D-Pip, L-NMe-Phe, and D-NMe-Val, wherein the phenyl group of L-NMe-Phe is optionally substituted by one or more substituents independently selected from halo, —SCH₃, —SOCH₃, —SO₂CH₃, —OH, —CN, —NO₂, C₁₋₄ alkyl, —CH₃, —CF₃, —CHF₂, —OBz, —OCH₃, —OCF₃, and —OCHF₂. In some embodiments, the first beta hairpin region comprises two contiguous residues independently selected from: L-Pro, D-Pro, L-Aze, D-Pip, and D-NMe-Val. In some embodiments, for the two contiguous residues, one is D and the other is L. In some embodiments, the two contiguous amino acid residues are D-Pro and L-Pro. In some embodiments, the two contiguous amino acid residues are D-NMe-Val and L-Pro. In some embodiments, the two contiguous amino acid residues are D-Pro and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the two contiguous amino acid residues are D-Pro and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂.

In some embodiments, the second beta hairpin region comprises a second two contiguous amino acid residues. In some embodiments, the second beta hairpin region comprises a second two contiguous residues independently selected from: D-Pro, a peptoid (e.g., sarcosine, N-isopropylglycine, N-benzylglycine, N-2-(methoxyethyl)glycine, etc.), a D-N-alkylated amino acid, and an L-N-alkylated amino acid. In some embodiments, the second beta hairpin region comprises a second two contiguous residues independently selected from: D-Pro, a peptoid, and an L-N-alkylated amino acid. In some embodiments, for the second two contiguous residues, one is a peptoid and the other is an L-N-alkylated amino acid. In some embodiments, for the second two contiguous residues, one is L-NMe-Ala and the other is N-(2-methoxyethyl)glycine. In some embodiments, for the second two contiguous residues, one is a D-N-alkylated amino acid and the other is an L-N-alkylated amino acid. In some embodiments, for the second two contiguous residues, one is D-NMe-Ala and the other is L-NMe-Ala. In some embodiments, for the second two contiguous residues, one is a D-N-alkylated amino acid and the other is a peptoid. In some embodiments, for the second two contiguous residues, one is D-NMe-Ala and the other is N-(2-methoxyethyl)glycine.

In some embodiments, at least two contiguous amino acids separate the first beta hairpin region from the second beta hairpin region. In some embodiments, at least three contiguous amino acids separate the first beta hairpin region from the second beta hairpin region.

In some embodiments, the molecular weight of the cyclic peptide is from 800 to 1300 Da. In some embodiments, the molecular weight of the cyclic peptide is from 800 to 1200 Da. In some embodiments, the molecular weight of the cyclic peptide is from 900 to 1200 Da.

In some embodiments, the cyclic peptide is characterized by at least four amino acid residues comprising rings independently selected from optionally substituted monocyclic carbocycle and optionally substituted monocyclic heterocycle, wherein at least one of the monocyclic carbocycle and monocyclic heterocycle are substituted. In some embodiments, the optionally substituted monocyclic carbocycle is phenyl and optionally substituted monocyclic heterocycle is a heteroaryl ring, wherein at least one phenyl or heteroaryl ring is substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the optionally substituted monocyclic carbocycle is phenyl and optionally substituted monocyclic heterocycle is a heteroaryl ring, wherein at least one phenyl or heteroaryl ring is substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the optionally substituted monocyclic carbocycle is phenyl and the optionally substituted monocyclic heterocycle is a heteroaryl ring, wherein at least one phenyl or heteroaryl ring is substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which may be substituted.

In some embodiments, the cyclic peptide is characterized by at least four amino acid residues with side chains selected from -alkylene-(monocyclic carbocycle) and -alkylene-(monocyclic heterocycle), wherein the monocyclic carbocycle and monocyclic heterocycle are independently optionally substituted. In some embodiments, each of the at least four amino acids with side chains selected from -alkylene-(optionally substituted monocyclic carbocycle) and -alkylene-(optionally substituted monocyclic heterocycle) are not adjacent to one another. In some embodiments, two of the at least four amino acids with side chains selected from -alkylene-(optionally substituted monocyclic carbocycle) and -alkylene-(optionally substituted monocyclic heterocycle) are adjacent to one another. In some embodiments, each monocyclic carbocycle is phenyl and each monocyclic heterocycle is a heteroaryl ring, wherein each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, each monocyclic carbocycle is phenyl and each monocyclic heterocycle is a heteroaryl ring, wherein each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, each monocyclic carbocycle is phenyl and each monocyclic heterocycle is a heteroaryl ring, wherein each phenyl and heteroaryl ring is independently optionally substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, and —OCHF₂.

In some embodiments, the cyclic peptide is characterized by at least three amino acid residues comprising rings independently selected from optionally substituted phenyl and optionally substituted monocyclic heteroaryl. In some embodiments, each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, each phenyl and heteroaryl ring is independently optionally substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, and —OCHF₂.

In some embodiments, at least three backbone nitrogen atoms of the cyclic peptide are tertiary nitrogens. In some embodiments, four or five backbone nitrogen atoms of the cyclic peptide are tertiary nitrogens. In some embodiments, four backbone nitrogen atoms of the cyclic peptide are tertiary nitrogens. In some embodiments, five backbone nitrogen atoms of the cyclic peptide are tertiary nitrogens. In some embodiments, one or more of the tertiary backbone nitrogen atoms are part of a heterocycloalkyl ring. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —SCH₃, —SOCH₃, —SO₂CH₃, —OH, —CN, —NO₂, C₁₋₄alkyl, —OBz, —OCH₃, —OCF₃, —SF₅, and —OCHF₂. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —SCH₃, —SOCH₃, —SO₂CH₃, —OH, —CN, —NO₂, C₁₋₄alkyl, —OBz, —OCH₃, —OCF₃, and —OCHF₂. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, each tertiary nitrogen is independently represented by:

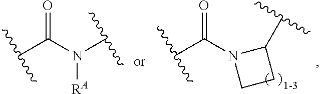

wherein R^{A} is C₁-C₆ alkyl optionally substituted with one or more substituents independently selected from halo, —SCH₃, —SOCH₃, —SO₂CH₃, —OH, —CN, —NO₂, C₁₋₄alkyl, —OBz, —OCH₃, —OCF₃, and —OCHF₂ and wherein

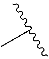

represents the point of connectivity to an adjacent amino acid residue. In some embodiments, each tertiary nitrogen is independently represented by:

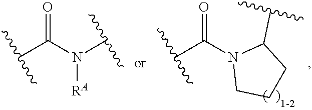

wherein R^{A} is C₁-C₆ alkyl optionally substituted with one or more substituents independently selected from halo, —OBz, —OCH₃, —OCF₃, and —OCHF₂ and wherein

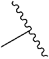

represents the point of connectivity to an adjacent amino acid residue.

In some embodiments, the cyclic peptide has 10 amino acid residues.

In some embodiments, the cyclic peptide is represented by Formula I:

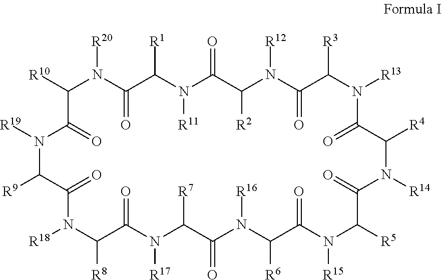

wherein:

R¹, R⁶, and R⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂;

R² is selected from hydrogen and C₁₋₆alkyl;

R³ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁴ is hydrogen or C₁₋₄alkyl, or R⁴ and R¹⁴ are taken together with the intervening atoms to form a 4- to 7-membered heterocycloalkyl;

R⁵ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂; or R⁵ and R¹⁵ are taken together with the intervening atoms to form a 4- to 7-membered heterocycloalkyl;

R⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, —SF₅, and —OCHF₂, or R⁷ and R¹⁷ are taken together with the intervening atoms to form a 4- to 7-membered heterocycloalkyl;

R⁹ is hydrogen or C₁₋₆alkyl, or R⁹ and R¹⁹ are taken together with the intervening atoms to form a 4- to 7-membered heterocycloalkyl;

R¹⁰ is hydrogen or C₁₋₄alkyl, or R¹⁰ and R²⁰ are taken together with the intervening atoms to form a 4- to 7-membered heterocycloalkyl;

R¹¹, R¹², R¹³, R¹⁶ and R¹⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R¹⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁴ and R⁴ are taken together with the intervening atoms to form a 4- to 7-membered heterocycloalkyl;

R¹⁵ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁵ and R⁵ are taken together with the intervening atoms to form a 4- to 7-membered heterocycloalkyl;

R¹⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁷ and R⁷ are taken together with the intervening atoms to form a 4- to 7-membered heterocycloalkyl;

R¹⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁹ and R⁹ are taken together with the intervening atoms to form a 4- to 7-membered heterocycloalkyl; and

R²⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂ or R²⁰ and R¹⁰ are taken together with the intervening atoms to form a 4- to 7-membered heterocycloalkyl.

In some embodiments, the cyclic peptide is represented by Formula I:

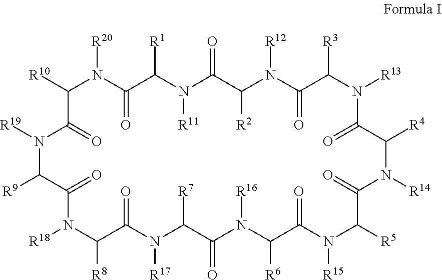

wherein:

R¹, R⁶, and R⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂;

R² is selected from hydrogen and C₁₋₆alkyl;

R³ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁴ is hydrogen or C₁₋₄alkyl, or R⁴ and R¹⁴ are taken together with the intervening atoms to form a 4- to 7-membered heterocycloalkyl;

R⁵ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂; or R⁵ and R¹⁵ are taken together with the intervening atoms to form a 4- to 7-membered heterocycloalkyl;

R⁷ is hydrogen or C₁₋₆alkyl, or R⁷ and R¹⁷ are taken together with the intervening atoms to form a 4- to 7-membered heterocycloalkyl;

R⁹ is hydrogen or C₁₋₆alkyl, or R⁹ and R¹⁹ are taken together with the intervening atoms to form a 4- to 7-membered heterocycloalkyl;

R¹⁰ is hydrogen or C₁₋₄alkyl, or R¹⁰ and R²⁰ are taken together with the intervening atoms to form a 4- to 7-membered heterocycloalkyl;

R¹¹, R¹², R¹³, R¹⁶, and R¹⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R¹⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁴ and R⁴ are taken together with the intervening atoms to form a 4- to 7-membered heterocycloalkyl;

R¹⁵ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁵ and R⁵ are taken together with the intervening atoms to form a 4- to 7-membered heterocycloalkyl;

R¹⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁷ and R⁷ are taken together with the intervening atoms to form a 4- to 7-membered heterocycloalkyl;

R¹⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁹ and R⁹ are taken together with the intervening atoms to form a 4- to 7-membered heterocycloalkyl; and

R²⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂ or R²⁰ and R¹⁰ are taken together with the intervening atoms to form a 4- to 7-membered heterocycloalkyl.

In some embodiments, the cyclic peptide is represented by Formula II:

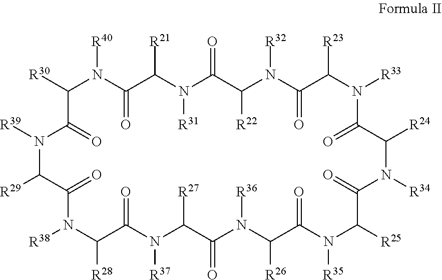

wherein:

R²¹, R²³, R²⁶, and R²⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —OCF₃, —SF₅, and —OCHF₂;

R²⁴ is hydrogen or C₁₋₄alkyl, or R²⁴ and R³⁴ are taken together with the intervening atoms to form a 5- to 7-membered heterocycloalkyl;

R²⁵ is hydrogen or C₁₋₄alkyl, or R²⁵ and R³⁵ are taken together with the intervening atoms to form a 5- to 7-membered heterocycloalkyl;

R³⁰ is hydrogen or C₁₋₄alkyl, or R³⁰ and R⁴⁰ are taken together with the intervening atoms to form a 5- to 7-membered heterocycloalkyl;

R²², R²⁷, and R²⁹ are independently selected from hydrogen and C₁₋₆alkyl;

R³¹, R³², R³³, R³⁶, and R³⁸ are independently selected from hydrogen; and C₁₋₄alkyl optionally substituted with one or more substituents independently selected from halo, —OH, —OBz, —OCH₃, —OCF₃, —SF₅, and —OCHF₂;

R³⁷ and R³⁹ are independently selected from hydrogen; and C₁₋₄alkyl optionally substituted with one or more substituents independently selected from halo, —OH, —OBz, —OCH₃, —OCF₃, —SF₅, and —OCHF₂;

R³⁴ is selected from hydrogen; and C₁₋₄alkyl optionally substituted with one or more substituents independently selected from halo, —OH, —OBz, —OCH₃, —OCF₃, —SF₅, and —OCHF₂, or R³⁴ and R²⁴ are taken together with the intervening atoms to form a 5- to 7-membered heterocycloalkyl;

R³⁵ is selected from hydrogen; and C₁₋₄alkyl optionally substituted with one or more substituents independently selected from halo, —OH, —OBz, —OCH₃, —OCF₃, —SF₅, and —OCHF₂, or R³⁵ and R²⁵ are taken together with the intervening atoms to form a 5- to 7-membered heterocycloalkyl; and

R⁴⁰ is selected from hydrogen; and C₁₋₄alkyl optionally substituted with one or more substituents independently selected from halo, —OH, —OBz, —OCH₃, —OCF₃, —SF₅, and —OCHF₂ or R⁴⁰ and R³⁰ are taken together with the intervening atoms to form a 5- to 7-membered heterocycloalkyl.

In some embodiments, the cyclic peptide is represented by Formula II:

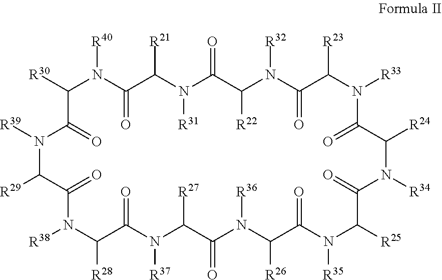

wherein:

R²¹, R²³, R²⁶, and R²⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —OCF₃, and —OCHF₂;

R²⁴ is hydrogen or C₁₋₄alkyl, or R²⁴ and R³⁴ are taken together with the intervening atoms to form a 5- to 7-membered heterocycloalkyl;

R²⁵ is hydrogen or C₁₋₄alkyl, or R²⁵ and R³⁵ are taken together with the intervening atoms to form a 5- to 7-membered heterocycloalkyl;

R³⁰ is hydrogen or C₁₋₄alkyl, or R³⁰ and R⁴⁰ are taken together with the intervening atoms to form a 5- to 7-membered heterocycloalkyl;

R²², R²⁷, and R²⁹ are independently selected from hydrogen and C₁₋₆alkyl;

R³¹, R³², R³³, R³⁶, and R³⁸ are independently selected from hydrogen; and C₁₋₄alkyl optionally substituted with one or more substituents independently selected from halo, —OH, —OBz, —OCH₃, —OCF₃, and —OCHF₂;

R³⁷ and R³⁹ are independently selected from hydrogen; and C₁₋₄alkyl optionally substituted with one or more substituents independently selected from halo, —OH, —OBz, —OCH₃, —OCF₃, and —OCHF₂;

R³⁴ is selected from hydrogen; and C₁₋₄alkyl optionally substituted with one or more substituents independently selected from halo, —OH, —OBz, —OCH₃, —OCF₃, and —OCHF₂, or R³⁴ and R²⁴ are taken together with the intervening atoms to form a 5- to 7-membered heterocycloalkyl;

R³⁵ is selected from hydrogen; and C₁₋₄alkyl optionally substituted with one or more substituents independently selected from halo, —OH, —OBz, —OCH₃, —OCF₃, and —OCHF₂, or R³⁵ and R²⁵ are taken together with the intervening atoms to form a 5- to 7-membered heterocycloalkyl; and

R⁴⁰ is selected from hydrogen; and C₁₋₄alkyl optionally substituted with one or more substituents independently selected from halo, —OH, —OBz, —OCH₃, —OCF₃, and —OCHF₂ or R⁴⁰ and R³⁰ are taken together with the intervening atoms to form a 5- to 7-membered heterocycloalkyl.

In some embodiments, R³¹, R³², R³³, R³⁶, and R³⁸ are each hydrogen.

In some embodiments, at least four of R³⁴, R³⁵, R³⁷, R³⁹, and R⁴⁰ are not hydrogen. In some embodiments, four of R³⁴, R³⁵, R³⁷, R³⁹, and R⁴⁰ are not hydrogen. In some embodiments, R³⁴, R³⁵, R³⁷, R³⁹, and R⁴⁰ are not hydrogen.

In some embodiments, at least one of R²⁴ and R³⁴, R²⁵ and R³⁵, and R³⁰ and R⁴⁰ are taken together with the intervening atoms to form a 5- to 7-membered heterocycloalkyl. In some embodiments, R²⁴ and R³⁴ are taken together with the intervening atoms to form a 5- to 6-membered heterocycloalkyl. In some embodiments, R²⁵ and R³⁵ are taken together with the intervening atoms to form a 5- to 6-membered heterocycloalkyl.

In some embodiments, each of R³⁷, R³⁹, and R⁴⁰ is selected from methyl and methoxyethyl. In some embodiments, each of R³⁵, R³⁷, R³⁹, and R⁴⁰ is selected from methyl and methoxyethyl.

In some embodiments, R⁴⁰ is C₂₋₄alkyl optionally substituted with one or more substituents independently selected from halo, —OH, —OBz, —OCH₃, —OCF₃, and —OCHF₂ or R³⁰ and R⁴⁰ are taken together with the intervening atoms to form a 5- to 7-membered heterocycloalkyl. In some embodiments, R⁴⁰ is C₂₋₄alkyl optionally substituted with one or more substituents independently selected from halo, —OH, —OBz, —OCH₃, —OCF₃, —SF₅, and —OCHF₂. In some embodiments, R⁴⁰ is C₂₋₄alkyl optionally substituted with one or more substituents independently selected from halo, —OH, —OBz, —OCH₃, —OCF₃, and —OCHF₂.

In some embodiments, R³⁹ is C₁₋₄alkyl optionally substituted with one or more substituents independently selected from halo, —OH, —OBz, —OCH₃, —OCF₃, and —OCHF₂.

In some embodiments, R²², R²⁷, and R²⁹ are independently selected from C₁₋₆alkyl. In some embodiments, R²², R²⁷, and R²⁹ are selected from methyl, ethyl, propyl, i-propyl, butyl, i-butyl, and t-butyl.

In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted. In some embodiments, R²¹, R²³, R²⁶, and R²⁸ are independently selected from —CH₂—(C₃₋₈carbocycle), and —CH₂-(3-10 membered heterocycle). In some embodiments, R²¹, R²³, R²⁶, and R²⁸ are independently selected from phenylmethyl and pyridinylmethyl, wherein the phenyl and pyridinyl are optionally substituted. In some embodiments, R²¹, R²³, R²⁶, and R²⁸ are independently selected from:

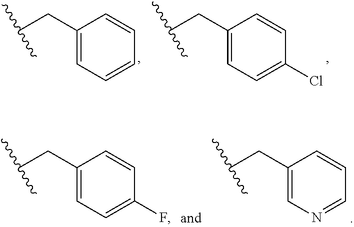

In some embodiments, the cyclic peptide is represented by Formula IIa:

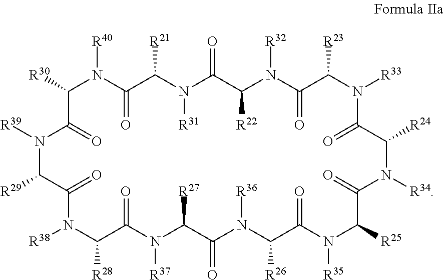

In some embodiments, the cyclic peptide is represented by Formula IIb:

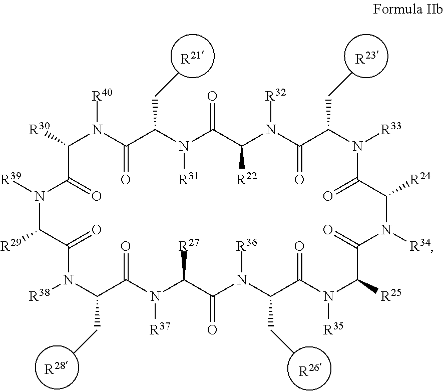

wherein R^{21′}, R^{23′}, R^{26′} and R^{28′} are independently selected from optionally substituted phenyl and optionally substituted 5- or 6-membered heteroaryl.

In some embodiments, the cyclic peptide is represented by Formula III:

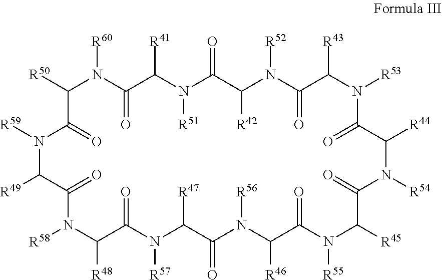

wherein:

R⁴¹, R⁴⁵, R⁴⁶, and R⁴⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂;

R⁴² is selected from hydrogen and C₁₋₆alkyl;

R⁴³ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁴⁴ is hydrogen or C₁₋₄alkyl, or R⁴⁴ and R⁵⁴ are taken together with the intervening atoms to form a 4- to 7-membered heterocycloalkyl;

R⁴⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, —SF₅, and —OCHF₂, or R⁴⁷ and R⁵⁷ are taken together with the intervening atoms to form a 4- to 7-membered heterocycloalkyl;

R⁴⁹ is hydrogen or C₁₋₆alkyl, or R⁴⁹ and R⁵⁹ are taken together with the intervening atoms to form a 4- to 7-membered heterocycloalkyl;

R⁵⁰ is hydrogen or C₁₋₄alkyl, or R⁵⁰ and R⁶⁰ are taken together with the intervening atoms to form a 4- to 7-membered heterocycloalkyl;

R⁵¹, R⁵³, R⁵⁶, and R⁵⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁵² and R⁵⁵ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁵⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁴ and R⁴⁴ are taken together with the intervening atoms to form a 4- to 7-membered heterocycloalkyl;

R⁵⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁷ and R⁴⁷ are taken together with the intervening atoms to form a 4- to 7-membered heterocycloalkyl;

R⁵⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁹ and R⁴⁹ are taken together with the intervening atoms to form a 4- to 7-membered heterocycloalkyl;

R⁶⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁶⁰ and R⁵⁰ are taken together with the intervening atoms to form a 4- to 7-membered heterocycloalkyl.

In some embodiments, the cyclic peptide is represented by Formula III:

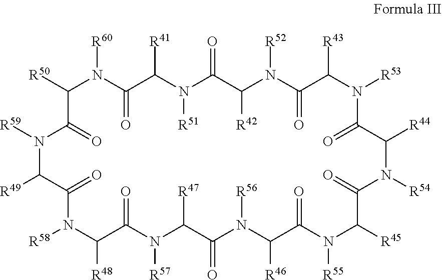

wherein:

R⁴¹, R⁴⁵, R⁴⁶, and R⁴⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂;

R⁴² is selected from hydrogen and C₁₋₆alkyl;

R⁴³ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁴⁴ is hydrogen or C₁₋₄alkyl, or R⁴⁴ and R⁵⁴ are taken together with the intervening atoms to form a 4- to 7-membered heterocycloalkyl;

R⁴⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, and —OCHF₂, or R⁴⁷ and R⁵⁷ are taken together with the intervening atoms to form a 4- to 7-membered heterocycloalkyl;

R⁴⁹ is hydrogen or C₁₋₆alkyl, or R⁴⁹ and R⁵⁹ are taken together with the intervening atoms to form a 4- to 7-membered heterocycloalkyl;

R⁵⁰ is hydrogen or C₁₋₄alkyl, or R⁵⁰ and R⁶⁰ are taken together with the intervening atoms to form a 4- to 7-membered heterocycloalkyl;

R⁵¹, R⁵³, R⁵⁶, and R⁵⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁵² and R⁵⁵ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁵⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁴ and R⁴⁴ are taken together with the intervening atoms to form a 4- to 7-membered heterocycloalkyl;

R⁵⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁷ and R⁴⁷ are taken together with the intervening atoms to form a 4- to 7-membered heterocycloalkyl;

R⁵⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁹ and R⁴⁹ are taken together with the intervening atoms to form a 4- to 7-membered heterocycloalkyl;

R⁶⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁶⁰ and R⁵⁰ are taken together with the intervening atoms to form a 4- to 7-membered heterocycloalkyl.

In some embodiments, R⁵¹, R⁵³, R⁵⁶, and R⁵⁸ are each hydrogen.

In some embodiments, at least four of R⁵², R⁵⁴, R⁵⁵, R⁵⁷, R⁵⁹, and R⁶⁰ are not hydrogen.

In some embodiments, four of R², R⁵⁴, R⁵⁵, R⁵⁷, R⁵⁹, and R⁶⁰ are not hydrogen. In some embodiments, R⁵², R⁵⁴, R⁵⁵, R⁵⁷, R⁵⁹, and R⁶⁰ are not hydrogen.

In some embodiments, at least one of R⁴⁴ and R⁵⁴, and R⁵⁰ and R⁶⁰ are taken together with the intervening atoms to form a 4- to 7-membered heterocycloalkyl. In some embodiments, R⁴⁴ and R⁵⁴ are taken together with the intervening atoms to form a 4- to 6-membered heterocycloalkyl.

In some embodiments, each of R⁵⁵, R⁵⁹, and R⁶⁰ is selected from methyl, ethyl, and methoxyethyl. In some embodiments, each of R⁵², R⁵⁵, R⁵⁹, and R⁶⁰ is selected from methyl, ethyl, and methoxyethyl.

In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂.

In some embodiments, R⁵⁹ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂.

In some embodiments, R⁴², R⁴⁷, and R⁴⁹ are independently selected from C₁₋₆alkyl. In some embodiments, R⁴², R⁴⁷, and R⁴⁹ are selected from methyl, ethyl, propyl, i-propyl, butyl, i-butyl, and t-butyl.

In some embodiments, R⁴¹, R⁴⁵, R⁴⁶, and R⁴⁸ are independently selected from —(C₁₋₄alkylene) —(C₃₋₈carbocycle) and —(C₁₋₄alkylene)-(3-10 membered heterocycle), wherein the C₃₋₈carbocycle and 3-10 membered heterocycle are optionally substituted. In some embodiments, R⁴¹, R⁴⁵, R⁴⁶, and R⁴⁸ are independently selected from —CH₂—(C₃₋₈carbocycle), and —CH₂-(3-10 membered heterocycle). In some embodiments, R⁴¹, R⁴⁵, R⁴⁶, and R⁴⁸ are independently selected from phenylmethyl, pyridinylmethyl, and thiazolylmethyl, wherein the phenyl, pyridinyl, and thiazolyl are optionally substituted. In some embodiments, R⁴¹, R⁴⁵, R⁴⁶, and R⁴⁸ are independently selected from:

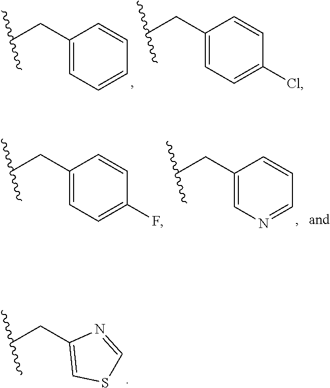

In some embodiments, the cyclic peptide is represented by Formula IIIa:

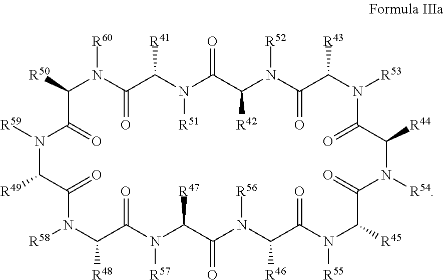

In some embodiments, the cyclic peptide is represented by Formula IIIb:

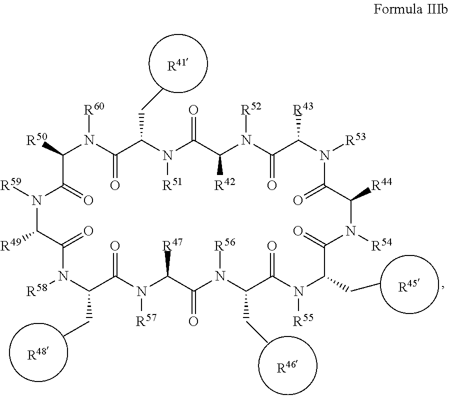

wherein R^{41′}, R^{45′}, R^{46′} and R^{48′} are independently selected from optionally substituted phenyl and optionally substituted 5- or 6-membered heteroaryl.

In some embodiments, the cyclic peptide is selected from those in Table 3 and Table 4, or a pharmaceutically acceptable salt of any one thereof.

In another aspect, the present disclosure provides a pharmaceutical composition comprising a cyclic peptide described herein and a pharmaceutically acceptable excipient.

In another aspect, the present disclosure provides a method of inhibiting MDM2, comprising administering a cyclic peptide described herein to a subject in need thereof.

In another aspect, the present disclosure provides a method of inhibiting MDM2 and MDM4, comprising administering a cyclic peptide described herein to a subject in need thereof.

In another aspect, the present disclosure provides a method of treating a disease or disorder in a subject in need thereof, comprising administering to said subject a therapeutically effective amount of a cyclic peptide described herein.

In some embodiments, the disease or disorder is cancer. In some embodiments, the cancer is selected from acute lymphoblastic leukemia, acute myeloid leukemia, chronic lymphocytic leukemia, and chronic myeloid leukemia. In some embodiments, the disease or disorder is associated with the proliferation of senescent cells. In some embodiments, the disease or disorder is selected from type 2 diabetes, Huntington's disease, non-alcoholic fatty liver disease, and hyperlipidemia. In some embodiments, the disease or disorder is selected from a cardiovascular disease, an inflammatory disease, an auto-immune disease, a metabolic disease, a pulmonary disease, an ophthalmic disease, an otic disease, a renal disease, and a dermatological disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the change in average tumor volume over time following intravenous administration of Compound 35 in a MOLM-13 mouse xenograft model.

FIG. 2 shows tumor volume at treatment day 13 following intravenous administration of Compound 35 in a MOLM-13 mouse xenograft model.

FIG. 3 shows the change in tumor volume over time following intravenous administration of Compound 35 in a MOLM-13 mouse xenograft model.

FIG. 4 shows the change in body weight over time following intravenous administration of Compound 35 in a MOLM-13 mouse xenograft model.

FIG. 5 shows the change in mean plasma concentration over time following intravenous administration of Compound 35 in a MOLM-13 mouse xenograft model.

### DETAILED DESCRIPTION

Mouse double minute 2 homolog (MDM2) and mouse double minute 4 homolog (MDM4) have shown promise as therapeutic targets for the treatment of various cancers. MDM2 and MDM4 are negative regulators of the p53 tumor suppressor gene via both E3 ubiquitin ligase activity and inhibition of p53 transcriptional activation. Further, because disruption of the protein-protein interaction between p53 and MDM2 or MDM4 can result in the death of senescent cells, the development of MDM2 and MDM4 inhibitors presents an opportunity for the treatment of diseases or disorders associated with the proliferation of senescent cells. A wide variety of diseases are associated with senescence, including cardiovascular diseases, inflammatory diseases, auto-immune diseases, metabolic diseases, pulmonary diseases, ophthalmic diseases, otic diseases, renal diseases, and dermatological diseases. Specific examples include type 2 diabetes, Huntington's disease, non-alcoholic fatty liver disease, and hyperlipidemia.

Cyclic peptides have emerged as potentially useful MDM2 and/or MDM4 inhibitors. Small molecule inhibitors of the MDM2/p53 protein-protein interaction and/or the MDM4/p53 protein-protein interaction are attractive as potential therapeutics for cancer. Beta hairpin regions are frequently found in nature as a means to display residues essential to protein-protein recognition. These beta hairpin regions of natural proteins can be simulated by carefully designed cyclic peptides, making cyclic peptides potentially useful as inhibitors of difficult to access targets, such as MDM2 and MDM4.

Despite their promise as therapeutic agents, the utility of cyclic peptides can be limited by poor pharmacokinetic properties, particularly poor cellular permeability, low solubility, and high clearance. There is a need for MDM2 inhibitors and MDM2/MDM4 dual inhibitors with improved pharmacokinetic properties such as improved cellular permeability for treating disease.

The present disclosure describes cyclic peptides which overcome the pharmacokinetic challenges of poor solubility and poor cell permeability. Specifically, the present disclosure provides cyclic peptides which have been optimized to enhance cell permeability and solubility.

Disclosed herein, in certain embodiments, are cyclic peptides useful as MDM2 inhibitors. In certain embodiments, the cyclic peptides disclosed herein are useful as MDM2/MDM4 dual inhibitors. In certain embodiments, cyclic peptides comprise nine to eleven amino acids independently selected from amino acid residues that are not charged at physiological pH, and a first and a second beta hairpin region. In certain embodiments, cyclic peptides are further characterized by one of the following: at least four amino acid residues comprising rings independently selected from optionally substituted monocyclic carbocycle and optionally substituted monocyclic heterocycle, wherein at least one of the monocyclic carbocycle and monocyclic heterocycle are substituted; at least four amino acid residues with side chains selected from -alkylene-(monocyclic carbocycle) and -alkylene-(monocyclic heterocycle), wherein the monocyclic carbocycle and monocyclic heterocycle are independently optionally substituted; and at least three amino acid residues comprising rings independently selected from optionally substituted phenyl and optionally substituted monocyclic heteroaryl.

In certain embodiments, the cyclic peptides disclosed herein display high cellular permeability and potent inhibition of MDM2 in both biochemical and cellular assays. In certain embodiments, the cyclic peptides disclosed herein display high cellular permeability and potent inhibition of MDM2 and MDM4 in both biochemical and cellular assays. In certain embodiments, the cyclic peptides disclosed herein hold therapeutic potential for the treatment of cancer.

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

As used herein, the abbreviations for amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Other amino acids include citrulline (Cit); homocysteine (Hey); hydroxyproline (Hyp); ornithine (Om); and thyroxine (Thx). Examples of amino acids that are not charged at physiological pH include, but are not limited to, alanine, asparagine, cysteine, glutamine, glycine, isoleucine, leucine, methionine, phenylalanine, proline, serine, threonine, tryptophan, tyrosine, and valine.

In the embodiments of the present disclosure, a cyclic peptide comprising a certain number of amino acid residues is a cyclic peptide wherein the cyclic peptide's backbone contains the recited number of amino acid residues. In other words, each of the amino acid residues is endocyclic. For example, for the purposes of this disclosure, the following would be considered a cyclic peptide comprising ten amino acid residues:

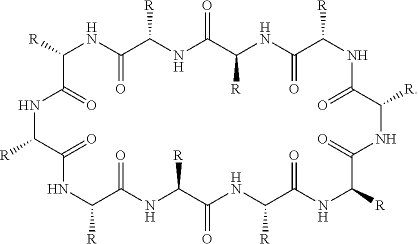

As another example, the following would also be considered a cyclic peptide comprising ten amino acid residues, not a cyclic peptide comprising eleven amino acid residues:

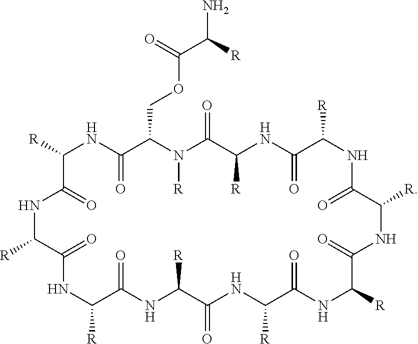

“Contiguous” amino acid residues are those endocyclic amino acids that are covalently bound in series without intervening endocyclic atoms. The following is an example of two contiguous proline residues wherein one is D and the other is L:

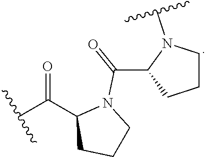 [SMILES: *C(=O)[C@@H]1CCCN1C(=O)[C@H]1CCCN1*]

In contrast, the following is an example of two proline residues that are not contiguous:

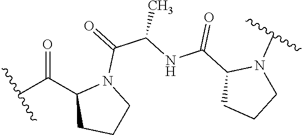 [SMILES: *C(=O)[C@@H]1CCCN1C(=O)[C@H](C)NC(=O)[C@H]1CCCN1*]

Where a number of contiguous amino acid residues separate a first and second beta hairpin region, e.g., at least three contiguous amino acids, the number refers to the number of residues starting from the C-terminus of the first beta hairpin region terminating at the N-terminus of the second beta hairpin region and/or the number of residues starting from the C-terminus of the second beta hairpin region terminating at the N-terminus of the first beta hairpin region. For example, the following illustrates an embodiment wherein the two beta hairpin regions are separated by three contiguous amino acid residues starting from the C-terminus of the first beta hairpin region terminating at the N-terminus of the second beta hairpin region and three contiguous amino acid residues starting from the C-terminus of the second beta hairpin region terminating at the N-terminus of the first beta hairpin region:

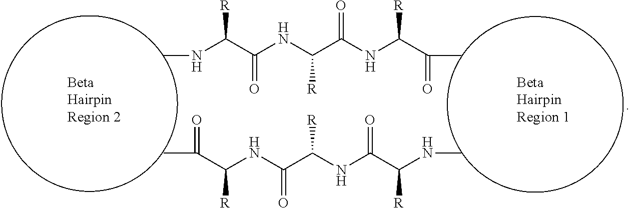

For another example, the following illustrates an embodiment wherein the two beta hairpin regions are separated by three contiguous amino acid residues starting from the C-terminus of the first beta hairpin region terminating at the N-terminus of the second beta hairpin region and two contiguous amino acid residues starting from the C-terminus of the second beta hairpin region terminating at the N-terminus of the first beta hairpin region:

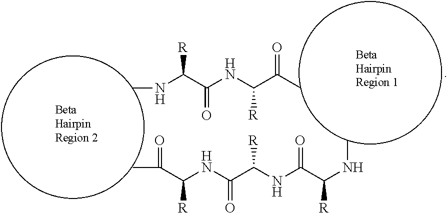

“Adjacent” residues are covalently bound to each other through an N- or C-terminus. Amino acid residues that are not adjacent to one another, have at least one amino acid or other atom separating the amino acid residues from the other on both the N-terminal and C-terminal sides. For example, for the following structure:

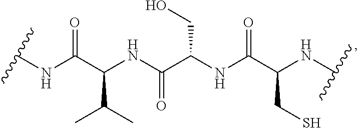 [SMILES: *NC(=O)[C@@H](NC(=O)[C@H](CO)NC(=O)[C@H](CS)N*)C(C)C]

the valine residue is adjacent to the serine residue, however the valine is not adjacent to the cysteine residue and the serine residue is adjacent to both the valine and cysteine residue.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene-refers to a substituted or unsubstituted alkylene chain with from x to y carbons in the alkylene chain. For example —C₁₋₆alkylene-may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. An alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, 6- to 12-membered bridged rings, and spirocyclic rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes, for example, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems.

The term “heteroaryl” as used herein refers to an aromatic ring comprising one or more heteroatoms. Exemplary monocyclic heteroaryl rings are 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, oxadiazole, thiazole, thiadiazole, triazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In certain aspects, the disclosure provides cyclic peptides. In one aspect, the present disclosure provides a cyclic peptide comprising:
nine to eleven amino acid residues independently selected from amino acid residues that are not charged at physiological pH;
a first and a second beta hairpin region; and characterized by one of the following:
at least four amino acid residues comprising rings independently selected from optionally substituted monocyclic carbocycle and optionally substituted monocyclic heterocycle, wherein at least one of the monocyclic carbocycle and monocyclic heterocycle are substituted;
at least four amino acid residues with side chains selected from -alkylene-(monocyclic carbocycle) and -alkylene-(monocyclic heterocycle), wherein the monocyclic carbocycle and monocyclic heterocycle are independently optionally substituted; and
at least three amino acid residues comprising rings independently selected from optionally substituted phenyl and optionally substituted monocyclic heteroaryl.

In some embodiments, the first beta hairpin region comprises two contiguous amino acid residues. In some embodiments, the first beta hairpin region comprises two contiguous residues independently selected from: L-Pro, D-Pro, L-Aze, D-Pip, L-NMe-Phe, and D-NMe-Val, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the first beta hairpin region comprises two contiguous residues independently selected from: L-Pro, D-Pro, L-Aze, D-Pip, L-NMe-Phe, and D-NMe-Val, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the first beta hairpin region comprises two contiguous residues independently selected from: L-Pro, D-Pro, L-Aze, D-Pip, and D-NMe-Val. In some embodiments, for the two contiguous residues, one is D and the other is L. In some embodiments, the two contiguous amino acid residues are D-Pro and L-Aze. In some embodiments, the two contiguous amino acid residues are D-Pro and L-Pro. In some embodiments, the two contiguous amino acid residues are D-Pro and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the two contiguous amino acid residues are D-Pro and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the two contiguous amino acid residues are D-Pip and L-Pro. In some embodiments, the two contiguous amino acid residues are D-Pip and L-Aze. In some embodiments, the two contiguous amino acid residues are D-Pip and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the two contiguous amino acid residues are D-Pip and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the two contiguous amino acid residues are D-NMe-Val and L-Pro. In some embodiments, the two contiguous amino acid residues are D-NMe-Val and L-Aze. In some embodiments, the two contiguous amino acid residues are D-NMe-Val and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the two contiguous amino acid residues are D-NMe-Val and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂.

In some embodiments, the second beta hairpin region comprises a second two contiguous amino acid residues. In some embodiments, the second beta hairpin region comprises a second two contiguous residues independently selected from: D-Pro, a peptoid, a D-N-alkylated amino acid, and an L-N-alkylated amino acid. In some embodiments, the second beta hairpin region comprises a second two contiguous residues independently selected from: D-Pro, a peptoid, and an L-N-alkylated amino acid. In some embodiments, for the second two contiguous residues, one is a peptoid and the other is an L-N-alkylated amino acid. In some embodiments, for the second two contiguous residues, one is L-NMe-Ala and the other is N-(2-methoxyethyl)glycine. In some embodiments, for the second two contiguous residues, one is D-Pro and the other is a peptoid. In some embodiments, for the second two contiguous residues, one is D-Pro and the other is an L-N-alkylated amino acid. In some embodiments, for the second two contiguous residues, one is D-Pro and the other is L-NMe-Ala. In some embodiments, for the second two contiguous residues, one is D-Pro and the other is N-(2-methoxyethyl)glycine. In some embodiments, for the second two contiguous residues, one is a D-N-alkylated amino acid and the other is an L-N-alkylated amino acid. In some embodiments, for the second two contiguous residues, one is D-NMe-Ala and the other is L-NMe-Ala. In some embodiments, for the second two contiguous residues, one is a D-N-alkylated amino acid and the other is a peptoid. In some embodiments, for the second two contiguous residues, one is D-NMe-Ala and the other is N-(2-methoxyethyl)glycine.

In some embodiments, at least two contiguous amino acids separate the first beta hairpin region from the second beta hairpin region. In some embodiments, at least three contiguous amino acids separate the first beta hairpin region from the second beta hairpin region. In some embodiments, two contiguous amino acids separate the first beta hairpin region from the second beta hairpin region. In some embodiments, three contiguous amino acids separate the first beta hairpin region from the second beta hairpin region. In certain embodiments, the number of contiguous amino acids between the first beta hairpin region and the second beta hairpin region refers to the number of amino acids starting from the C-terminus of the first beta hairpin region terminating at the N-terminus of the second beta hairpin region. In certain embodiments, the number of contiguous amino acids refers to the number of residues starting from the C-terminus of the second beta hairpin region terminating at the N-terminus of the first beta hairpin region. In certain embodiments, the number of contiguous amino acids refers the number of residues starting from the C-terminus of the first beta hairpin region terminating at the N-terminus of the second beta hairpin region and to the number of contiguous amino acids refers to the number of residues starting from the C-terminus of the second beta hairpin region terminating at the N-terminus of the first beta hairpin region, e.g., three contiguous amino acids starting from the C-terminus of the first beta hairpin region terminating at the N-terminus of the second beta hairpin region and three contiguous amino acids starting from the C-terminus of the second beta hairpin region terminating at the N-terminus of the first beta hairpin region.

In some embodiments, the molecular weight of the cyclic peptide is from 800 to 1300 Da. In some embodiments, the molecular weight of the cyclic peptide is from 800 to 1200 Da. In some embodiments, the molecular weight of the cyclic peptide is from 900 to 1200 Da. In some embodiments, the molecular weight of the cyclic peptide is from 800 to 900 Da. In some embodiments, the molecular weight of the cyclic peptide is from 900 to 1000 Da. In some embodiments, the molecular weight of the cyclic peptide is from 1000 to 1100 Da. In some embodiments, the molecular weight of the cyclic peptide is from 1100 to 1200 Da. In some embodiments, the molecular weight of the cyclic peptide is from 1200 to 1500 Da. In some embodiments, the molecular weight of the cyclic peptide is from 1200 to 1400 Da. In some embodiments, the molecular weight of the cyclic peptide is from 1100 to 1300 Da.

In some embodiments, the cyclic peptide has at least four amino acid residues comprising rings independently selected from optionally substituted monocyclic carbocycle and optionally substituted monocyclic heterocycle, wherein at least one of the monocyclic carbocycle and monocyclic heterocycle are substituted. In some embodiments, the at least four amino acid residues comprising rings independently selected from optionally substituted monocyclic carbocycle and optionally substituted monocyclic heterocycle are not adjacent to one another. In some embodiments, the cyclic peptide is characterized by four amino acid residues comprising rings independently selected from optionally substituted monocyclic carbocycle and optionally substituted monocyclic heterocycle, wherein at least one of the monocyclic carbocycle and monocyclic heterocycle are substituted. In some embodiments, the cyclic peptide is characterized by three amino acid residues comprising rings independently selected from optionally substituted monocyclic carbocycle and one amino acid residue comprising a ring independently selected from optionally substituted monocyclic heterocycle. In some embodiments, the optionally substituted monocyclic carbocycle is phenyl and optionally substituted monocyclic heterocycle is a heteroaryl ring, wherein at least one phenyl or heteroaryl ring is substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, the optionally substituted monocyclic carbocycle is phenyl and optionally substituted monocyclic heterocycle is a heteroaryl ring, wherein at least one phenyl or heteroaryl ring is substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the optionally substituted monocyclic carbocycle is phenyl and the optionally substituted monocyclic heterocycle is a heteroaryl ring, wherein at least one phenyl or heteroaryl ring is substituted by one or more substituents independently selected from halo, —CH₃, —CF₃, —CHF₂, —OBz, —OCH₃, —OCF₃, —SF₅, and —OCHF₂. In some embodiments, the optionally substituted monocyclic carbocycle is phenyl and the optionally substituted monocyclic heterocycle is a heteroaryl ring, wherein at least one phenyl or heteroaryl ring is substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which may be substituted. In some embodiments, the optionally substituted monocyclic carbocycle is phenyl and the optionally substituted monocyclic heterocycle is pyridine, wherein each ring is independently optionally substituted. In some embodiments, the at least four amino acid residues comprising rings independently selected from optionally substituted monocyclic carbocycle and optionally substituted monocyclic heterocycle are independently selected from phenylalanine, 3-(3-pyridyl)alanine, and 4-halophenylalanine.

In some embodiments, the cyclic peptide has at least four amino acid residues with side chains selected from -alkylene-(monocyclic carbocycle) and -alkylene-(monocyclic heterocycle), wherein the monocyclic carbocycle and monocyclic heterocycle are independently optionally substituted. In some embodiments, each of the at least four amino acids with side chains selected from -alkylene-(optionally substituted monocyclic carbocycle) and -alkylene-(optionally substituted monocyclic heterocycle) are not adjacent to one another. In some embodiments, two of the at least four amino acids with side chains selected from -alkylene-(optionally substituted monocyclic carbocycle) and -alkylene-(optionally substituted monocyclic heterocycle) are adjacent to one another. In some embodiments, each monocyclic carbocycle is phenyl and each monocyclic heterocycle is a heteroaryl ring, wherein each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, each monocyclic carbocycle is phenyl and each monocyclic heterocycle is a heteroaryl ring, wherein each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, the cyclic peptide is characterized by four amino acid residues with side chains selected from -alkylene-(monocyclic carbocycle) and -alkylene-(monocyclic heterocycle), wherein the monocyclic carbocycle and monocyclic heterocycle are independently optionally substituted. In some embodiments, the cyclic peptide is characterized by three amino acids with side chains independently selected from -alkylene-(monocyclic carbocycle) and one amino acid with a side chain selected from -alkylene-(monocyclic heterocycle), wherein the monocyclic carbocycles and monocyclic heterocycle are independently optionally substituted. In some embodiments, each monocyclic carbocycle is phenyl and each monocyclic heterocycle is a heteroaryl ring, wherein each phenyl and heteroaryl ring is independently optionally substituted by one or more substituents independently selected from halo, —CH₃, —CF₃, —CHF₂, —OBz, —OCH₃, —OCF₃, —SF₅, and —OCHF₂. In some embodiments, each monocyclic carbocycle is phenyl and each monocyclic heterocycle is a heteroaryl ring, wherein each phenyl and heteroaryl ring is independently optionally substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, —SF₅, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, and —OCHF₂. In some embodiments, each monocyclic carbocycle is phenyl and each monocyclic heterocycle is pyridine, wherein each ring is independently optionally substituted. In some embodiments, the at least four amino acid residues with side chains selected from -alkylene-(monocyclic carbocycle) and -alkylene-(monocyclic heterocycle) are independently selected from phenylalanine, 3-(3-pyridyl)alanine, and 4-halophenylalanine.

In some embodiments, the cyclic peptide has at least three amino acid residues comprising rings independently selected from optionally substituted phenyl and optionally substituted monocyclic heteroaryl. In some embodiments, the at least three amino acid residues comprising rings independently selected from optionally substituted phenyl and optionally substituted monocyclic heteroaryl are not adjacent to one another. In some embodiments, the cyclic peptide is characterized by three amino acid residues comprising rings independently selected from optionally substituted phenyl and optionally substituted monocyclic heteroaryl. In some embodiments, the cyclic peptide is characterized by four amino acid residues comprising rings independently selected from optionally substituted phenyl and optionally substituted monocyclic heteroaryl. In some embodiments, the cyclic peptide is characterized by three amino acid residues comprising rings independently selected from optionally substituted phenyl and one amino acid residue comprising a ring selected from optionally substituted monocyclic heteroaryl. In some embodiments, the cyclic peptide is characterized by three amino acid residues comprising rings independently selected from optionally substituted phenyl and one amino acid residue comprising a ring selected from optionally substituted pyridine. In some embodiments, each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂. In some embodiments, each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, and —OCHF₂. In some embodiments, each phenyl and heteroaryl ring is independently optionally substituted by one or more substituents independently selected from halo, —CH₃, —CF₃, —CHF₂, —OBz, —OCH₃, —OCF₃, —SF₅, and —OCHF₂. In some embodiments, each phenyl and heteroaryl ring is independently optionally substituted by one or more substituents independently selected from halo, —CH₃, —CF₃, —CHF₂, —OBz, —OCH₃, —OCF₃, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, —SF₅, and —OCHF₂. In some embodiments, each heteroaryl ring is independently selected from thiophene, thiazole, oxazole, triazole, tetrazole, pyridine, pyrimidine, pyrazine, pyrrole, pyrazole, and imidazole, any one of which is optionally substituted by one or more substituents independently selected from halo, —CH₃, —CF₃, —CHF₂, —OBz, —OCH₃, —OCF₃, and —OCHF₂. In some embodiments, each heteroaryl ring is independently optionally substituted pyridine. In some embodiments, the at least three amino acid residues comprising rings independently selected from optionally substituted phenyl and optionally substituted monocyclic heteroaryl are independently selected from phenylalanine, 3-(3-pyridyl)alanine, and 4-halophenylalanine.

In some embodiments, at least three backbone nitrogen atoms of the cyclic peptide are tertiary nitrogens. In some embodiments, four or five backbone nitrogen atoms of the cyclic peptide are tertiary nitrogens. In some embodiments, four backbone nitrogen atoms of the cyclic peptide are tertiary nitrogens. In some embodiments, five backbone nitrogen atoms of the cyclic peptide are tertiary nitrogens.

In some embodiments, one or more of the tertiary backbone nitrogen atoms are part of heterocycloalkyl ring(s). When two or more tertiary backbone nitrogen atoms are part of heterocycloalkyl rings, these rings are distinct from each other. For example, when there are two tertiary backbone nitrogen atoms part of heterocycloalkyl rings, one nitrogen is part of a first proline moiety and the second nitrogen is part of a second proline moiety.

In some embodiments, one tertiary backbone nitrogen atoms is part of a heterocycloalkyl ring. In some embodiments, one tertiary backbone nitrogen atom is part of a first heterocycloalkyl ring, and a second tertiary backbone nitrogen atom is part of a second heterocycloalkyl ring. In some embodiments, one tertiary backbone nitrogen atom is part of a first heterocycloalkyl ring, a second tertiary backbone nitrogen atom is part of a second heterocycloalkyl ring, and a third tertiary backbone nitrogen atom is part of a third heterocycloalkyl ring. In some embodiments, one tertiary backbone nitrogen atom is part of a first heterocycloalkyl ring, a second tertiary backbone nitrogen atom is part of a second heterocycloalkyl ring, a third tertiary backbone nitrogen atom is part of a third heterocycloalkyl ring, and a fourth tertiary backbone nitrogen atom is part of a fourth heterocycloalkyl ring. In some embodiments, one tertiary backbone nitrogen atom is part of a first heterocycloalkyl ring, a second tertiary backbone nitrogen atom is part of a second heterocycloalkyl ring, a third tertiary backbone nitrogen atom is part of a third heterocycloalkyl ring, a fourth tertiary backbone nitrogen atom is part of a fourth heterocycloalkyl ring, and a fifth tertiary backbone nitrogen atom is part of a fifth heterocycloalkyl ring.

In some embodiments, one or more of the tertiary nitrogens have an optionally substituted C₁-C₆ alkyl substituent independently selected at each tertiary nitrogen and wherein substituents on C₁-C₆ alkyl are independently selected from halo, —SCH₃, —SOCH₃, —SO₂CH₃, —OH, —CN, —NO₂, C₁₋₄alkyl, —OBz, —OCH₃, —OCF₃, —SF₅, and —OCHF₂. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —SCH₃, —SOCH₃, —SO₂CH₃, —OH, —CN, —NO₂, C₁₋₄alkyl, —OBz, —OCH₃, —OCF₃, and —OCHF₂. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, one or more of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, one of the tertiary nitrogens has an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, one of the tertiary nitrogens has an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, two of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, two of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, three of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, three of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, four of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, four of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂. In some embodiments, five of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, —SF₅, and —OCHF₂. In some embodiments, five of the tertiary nitrogens have an optionally substituted C₁-C₆ alkyl substituent independently selected at each tertiary nitrogen and wherein substituents on C₁-C₆ alkyl are independently selected from halo, —OBz, —OCH₃, —OCF₃, and —OCHF₂.

In some embodiments, each tertiary nitrogen is independently represented by:

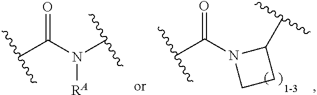

wherein R^{A} is C₁-C₆ alkyl optionally substituted with one or more substituents independently selected from halo, —SCH₃, —SOCH₃, —SO₂CH₃, —OH, —CN, —NO₂, C₁₋₄alkyl, —OBz, —OCH₃, —OCF₃, —SF₅, and —OCHF₂ and wherein

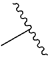

represents the point of connectivity to an adjacent amino acid residue. In some embodiments, each tertiary nitrogen is independently represented by:

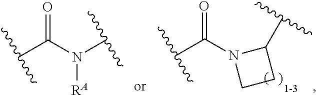

wherein R^{A} is C₁-C₆ alkyl optionally substituted with one or more substituents independently selected from halo, —SCH₃, —SOCH₃, —SO₂CH₃, —OH, —CN, —NO₂, C₁₋₄alkyl, —OBz, —OCH₃, —OCF₃, and —OCHF₂ and wherein

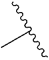

represents the point of connectivity to an adjacent amino acid residue. In some embodiments, each tertiary nitrogen is independently represented by:

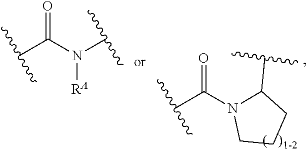

wherein R^{A} is C₁-C₆ alkyl optionally substituted with one or more substituents independently selected from halo, —OBz, —OCH₃, —OCF₃, —SF₅, and —OCHF₂ and wherein

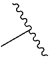

represents the point of connectivity to an adjacent amino acid residue. In some embodiments, each tertiary nitrogen is independently represented by:

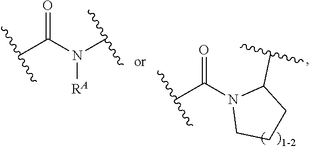

wherein R^{A} is C₁-C₆ alkyl optionally substituted with one or more substituents independently selected from halo, —OBz, —OCH₃, —OCF₃, and —OCHF₂ and wherein

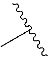

represents the point of connectivity to an adjacent amino acid residue. In some embodiments, one or more tertiary nitrogens is

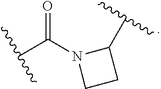 [SMILES: *C(=O)N1CCC1*]

In some embodiments, one or more tertiary nitrogens is

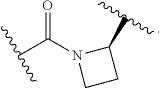 [SMILES: *C(=O)N1CC[C@@H]1*]

In some embodiments, one or more tertiary nitrogens is

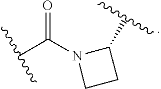 [SMILES: *C(=O)N1CC[C@H]1*]

In some embodiments, one or more tertiary nitrogens is

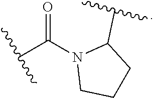 [SMILES: *C(=O)N1CCCC1*]

In some embodiments, one or more tertiary nitrogens is

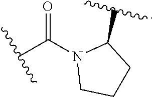 [SMILES: *C(=O)N1CCC[C@@H]1*]

In some embodiments, one or more tertiary nitrogens is

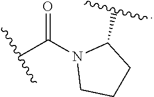 [SMILES: *C(=O)N1CCC[C@H]1*]

In some embodiments, one or more tertiary nitrogens is

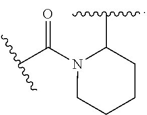 [SMILES: *C(=O)N1CCCCC1*]

In some embodiments, one or more tertiary nitrogens is

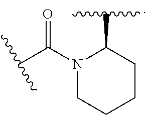 [SMILES: *C(=O)N1CCCC[C@@H]1*]

In some embodiments, one or more tertiary nitrogens is

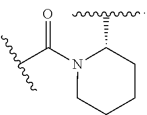 [SMILES: *C(=O)N1CCCC[C@H]1*]

In some embodiments, one or more tertiary nitrogens is

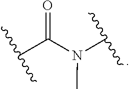 [SMILES: *C(=O)N(*)C]

In some embodiments, one or more tertiary nitrogens is

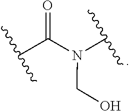 [SMILES: *C(=O)N(*)CO]

In some embodiments, one or more tertiary nitrogens is

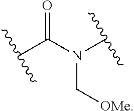 [SMILES: *C(=O)N(*)COC]

In some embodiments, one or more tertiary nitrogens is

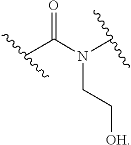 [SMILES: *C(=O)N(*)CCO]

In some embodiments, one or more tertiary nitrogens is

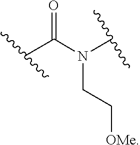 [SMILES: *C(=O)N(*)CCOC]

In some embodiments, the cyclic peptide has 8 amino acid residues. In some embodiments, the cyclic peptide has 9 amino acid residues. In some embodiments, the cyclic peptide has 10 amino acid residues. In some embodiments, the cyclic peptide has 11 amino acid residues. In some embodiments, the cyclic peptide has 12 amino acid residues.

In some embodiments, the cyclic peptide is represented by Formula I:

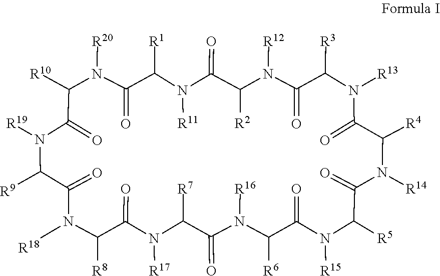

wherein:

R¹, R⁶, and R⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂;

R² is selected from hydrogen and C₁₋₆alkyl;

R³ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁴ is hydrogen or C₁₋₄alkyl, or R⁴ and R¹⁴ are taken together with the intervening atoms to form a 4- to 7-membered heterocycloalkyl;

R⁵ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂; or R⁵ and R¹⁵ are taken together with the intervening atoms to form a 4- to 7-membered heterocycloalkyl;

R⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, —SF₅, and —OCHF₂, or R⁷ and R¹⁷ are taken together with the intervening atoms to form a 4- to 7-membered heterocycloalkyl;

R⁹ is hydrogen or C₁₋₆alkyl, or R⁹ and R¹⁹ are taken together with the intervening atoms to form a 4- to 7-membered heterocycloalkyl;

R¹⁰ is hydrogen or C₁₋₄alkyl, or R¹⁰ and R²⁰ are taken together with the intervening atoms to form a 4- to 7-membered heterocycloalkyl;

R¹¹, R¹², R¹³, R¹⁶, and R¹⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R¹⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁴ and R⁴ are taken together with the intervening atoms to form a 4- to 7-membered heterocycloalkyl;

R¹⁵ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁵ and R⁵ are taken together with the intervening atoms to form a 4- to 7-membered heterocycloalkyl;

R¹⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁷ and R⁷ are taken together with the intervening atoms to form a 4- to 7-membered heterocycloalkyl;

R¹⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R¹⁹ and R⁹ are taken together with the intervening atoms to form a 4- to 7-membered heterocycloalkyl; and

R²⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂ or R²⁰ and R¹⁰ are taken together with the intervening atoms to form a 4- to 7-membered heterocycloalkyl.

In some embodiments, the cyclic peptide is represented by Formula I:

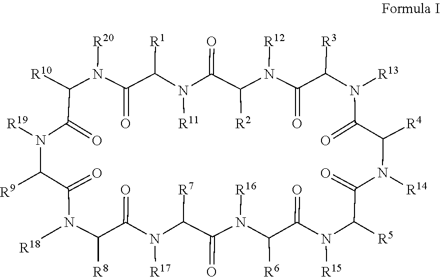

wherein:

R¹, R⁶, and R⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂;

R² is selected from hydrogen and C₁₋₆alkyl;

R³ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁴ is hydrogen or C₁₋₄alkyl, or R⁴ and R¹⁴ are taken together with the intervening atoms to form a 4- to 7-membered heterocycloalkyl;

R⁵ is selected from hydrogen, C₁₋₄alkyl, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂; and wherein the C₁₋₄alkyl is optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂; or R⁵ and R¹⁵ are taken together with the intervening atoms to form a 4- to 7-membered heterocycloalkyl;

R⁷ is hydrogen or C₁₋₆alkyl, or R⁷ and R¹⁷ are taken together with the intervening atoms to form a 4- to 7-membered heterocycloalkyl;

R⁹ is hydrogen or C₁₋₆alkyl, or R⁹ and R¹⁹ are taken together with the intervening atoms to form a 4- to 7-membered heterocycloalkyl;

R¹⁰ is hydrogen or C₁₋₄alkyl, or R¹⁰ and R²⁰ are taken together with the intervening atoms to form a 4- to 7-membered heterocycloalkyl;

R¹¹, R¹², R¹³, R¹⁶, and R¹⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R¹⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁴ and R⁴ are taken together with the intervening atoms to form a 4- to 7-membered heterocycloalkyl;

R¹⁵ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁵ and R⁵ are taken together with the intervening atoms to form a 4- to 7-membered heterocycloalkyl;

R¹⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁷ and R⁷ are taken together with the intervening atoms to form a 4- to 7-membered heterocycloalkyl;

R¹⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R¹⁹ and R⁹ are taken together with the intervening atoms to form a 4- to 7-membered heterocycloalkyl; and

R²⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂ or R²⁰ and R¹⁰ are taken together with the intervening atoms to form a 4- to 7-membered heterocycloalkyl.

In some embodiments, the cyclic peptide is represented by Formula II:

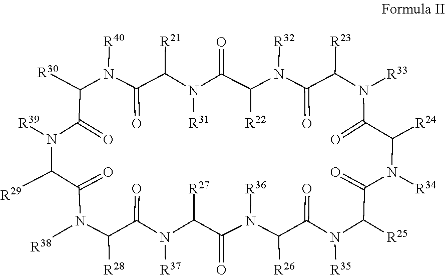

wherein:

R²¹, R²³, R²⁶, and R²⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —OCF₃, —SF₅, and —OCHF₂;

R²⁴ is hydrogen or C₁₋₄alkyl, or R²⁴ and R³⁴ are taken together with the intervening atoms to form a 5- to 7-membered heterocycloalkyl;

R²⁵ is hydrogen or C₁₋₄alkyl, or R²⁵ and R³⁵ are taken together with the intervening atoms to form a 5- to 7-membered heterocycloalkyl;

R³⁰ is hydrogen or C₁₋₄alkyl, or R³⁰ and R⁴⁰ are taken together with the intervening atoms to form a 5- to 7-membered heterocycloalkyl;

R²², R²⁷, and R²⁹ are independently selected from hydrogen and C₁₋₆alkyl;

R³¹, R³², R³³, R³⁶, and R³⁸ are independently selected from hydrogen; and C₁₋₄alkyl optionally substituted with one or more substituents independently selected from halo, —OH, —OBz, —OCH₃, —OCF₃, —SF₅, and —OCHF₂;

R³⁷ and R³⁹ are independently selected from hydrogen; and C₁₋₄alkyl optionally substituted with one or more substituents independently selected from halo, —OH, —OBz, —OCH₃, —OCF₃, —SF₅, and —OCHF₂;

R³⁴ is selected from hydrogen; and C₁₋₄alkyl optionally substituted with one or more substituents independently selected from halo, —OH, —OBz, —OCH₃, —OCF₃, —SF₅, and —OCHF₂, or R³⁴ and R²⁴ are taken together with the intervening atoms to form a 5- to 7-membered heterocycloalkyl;

R³⁵ is selected from hydrogen; and C₁₋₄alkyl optionally substituted with one or more substituents independently selected from halo, —OH, —OBz, —OCH₃, —OCF₃, —SF₅, and —OCHF₂, or

R³⁵ and R²⁵ are taken together with the intervening atoms to form a 5- to 7-membered heterocycloalkyl; and

R⁴⁰ is selected from hydrogen; and C₁₋₄alkyl optionally substituted with one or more substituents independently selected from halo, —OH, —OBz, —OCH₃, —OCF₃, —SF₅, and —OCHF₂ or R⁴⁰ and R³⁰ are taken together with the intervening atoms to form a 5- to 7-membered heterocycloalkyl.

In some embodiments, the cyclic peptide is represented by Formula II:

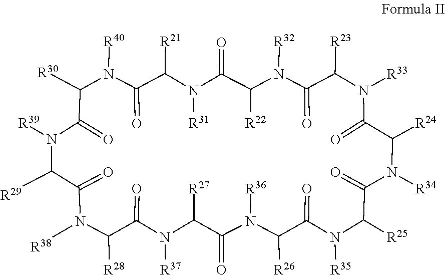

wherein:

R²¹, R²³, R²⁶, and R²⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —OCF₃, and —OCHF₂;

R²⁴ is hydrogen or C₁₋₄alkyl, or R²⁴ and R³⁴ are taken together with the intervening atoms to form a 5- to 7-membered heterocycloalkyl;

R²⁵ is hydrogen or C₁₋₄alkyl, or R²⁵ and R³⁵ are taken together with the intervening atoms to form a 5- to 7-membered heterocycloalkyl;

R³⁰ is hydrogen or C₁₋₄alkyl, or R³⁰ and R⁴⁰ are taken together with the intervening atoms to form a 5- to 7-membered heterocycloalkyl;

R²², R²⁷, and R²⁹ are independently selected from hydrogen and C₁₋₆alkyl;

R³¹, R³², R³³, R³⁶, and R³⁸ are independently selected from hydrogen; and C₁₋₄alkyl optionally substituted with one or more substituents independently selected from halo, —OH, —OBz, —OCH₃, —OCF₃, and —OCHF₂;

R³⁷ and R³⁹ are independently selected from hydrogen; and C₁₋₄alkyl optionally substituted with one or more substituents independently selected from halo, —OH, —OBz, —OCH₃, —OCF₃, and —OCHF₂;

R³⁴ is selected from hydrogen; and C₁₋₄alkyl optionally substituted with one or more substituents independently selected from halo, —OH, —OBz, —OCH₃, —OCF₃, and —OCHF₂, or R³⁴ and R²⁴ are taken together with the intervening atoms to form a 5- to 7-membered heterocycloalkyl;

R³⁵ is selected from hydrogen; and C₁₋₄alkyl optionally substituted with one or more substituents independently selected from halo, —OH, —OBz, —OCH₃, —OCF₃, and —OCHF₂, or R³¹ and R²⁵ are taken together with the intervening atoms to form a 5- to 7-membered heterocycloalkyl; and

R⁴⁰ is selected from hydrogen; and C₁₋₄alkyl optionally substituted with one or more substituents independently selected from halo, —OH, —OBz, —OCH₃, —OCF₃, and —OCHF₂ or R⁴⁰ and R³⁰ are taken together with the intervening atoms to form a 5- to 7-membered heterocycloalkyl.

In some embodiments, R³¹ is hydrogen. In some embodiments, R³² is hydrogen. In some embodiments, R³³ is hydrogen. In some embodiments, R³⁶ is hydrogen. In some embodiments, R³⁸ is hydrogen. In some embodiments, R³¹ and R³² are each hydrogen. In some embodiments, R³¹ and R³³ are each hydrogen. In some embodiments, R³¹ and R³⁶ are each hydrogen. In some embodiments, R³¹ and R³⁸ are each hydrogen. In some embodiments, R³² and R³³ are each hydrogen. In some embodiments, R³² and R³⁶ are each hydrogen. In some embodiments, R³² and R³⁸ are each hydrogen. In some embodiments, R³³ and R³⁶ are each hydrogen. In some embodiments, R³³ and R³⁸ are each hydrogen. In some embodiments, R³⁶ and R³⁸ are each hydrogen. In some embodiments, R³¹, R²¹, and R³³ are each hydrogen. In some embodiments, R³¹, R³², and R³⁶ are each hydrogen. In some embodiments, R³¹, R³², and R³⁸ are each hydrogen. In some embodiments, R³¹, R³³, and R³⁶ are each hydrogen. In some embodiments, R³¹, R³³, and R³⁸ are each hydrogen. In some embodiments, R³¹, R³⁶, and R³⁸ are each hydrogen. In some embodiments, R³², R³³, and R³⁶ are each hydrogen. In some embodiments, R³², R³³, and R³⁸ are each hydrogen. In some embodiments, R³², R³⁶, and R³⁸ are each hydrogen. In some embodiments, R³³, R³⁶, and R³⁸ are each hydrogen. In some embodiments, R³¹, R³², R³³, and R³⁶ are each hydrogen. In some embodiments, R³¹, R³², R³³, and R³⁸ are each hydrogen. In some embodiments, R³¹, R³³, R³⁶, and R³⁸ are each hydrogen. In some embodiments, R³¹, R³², R³⁶, and R³⁸ are each hydrogen. In some embodiments, R³², R³³, R³⁶, and R³⁸ are each hydrogen. In some embodiments, R³¹, R³², R³³, R³⁶, and R³⁸ are each hydrogen.

In some embodiments, at least four of R³⁴, R³⁵, R³⁷, R³⁹, and R⁴⁰ are not hydrogen. In some embodiments, four of R³⁴, R³⁵, R³⁷, R³⁹, and R⁴⁰ are not hydrogen. In some embodiments, R³⁴, R³⁵, R³⁷, and R³⁹ are not hydrogen. In some embodiments, R³⁴, R³⁵, R³⁷, and R⁴⁰ are not hydrogen. In some embodiments, R³⁵, R³⁷, R³⁹, and R⁴⁰ are not hydrogen. In some embodiments, R³⁴, R³⁵, R³⁹, and R⁴⁰ are not hydrogen. In some embodiments, R³⁴, R³⁷, R³⁹, and R⁴⁰ are not hydrogen. In some embodiments, R³⁴, R³⁵, R³⁷, R³⁹, and R⁴⁰ are not hydrogen.

In some embodiments, at least one of R²⁴ and R³⁴, R²⁵ and R³⁵, and R³⁰ and R⁴⁰ are taken together with the intervening atoms to form a 5- to 7-membered heterocycloalkyl. In some embodiments, at least two of R²⁴ and R³⁴, R²⁵ and R³⁵, and R³⁰ and R⁴⁰ are taken together with the intervening atoms to form a 5- to 7-membered heterocycloalkyl. In some embodiments, R²⁴ and R³⁴ are taken together with the intervening atoms to form a 5- to 6-membered heterocycloalkyl. In some embodiments, R²⁵ and R³⁵ are taken together with the intervening atoms to form a 5- to 6-membered heterocycloalkyl. In some embodiments, R³⁰ and R⁴⁰ are taken together with the intervening atoms to form a 5- to 6-membered heterocycloalkyl. In some embodiments, R²⁴ and R³⁴ and R²⁵ and R³⁵ are taken together with the intervening atoms to form a 5- to 6-membered heterocycloalkyl. In some embodiments, R²⁴ and R³⁴ and R³⁰ and R⁴⁰ are taken together with the intervening atoms to form a 5- to 6-membered heterocycloalkyl. In some embodiments, R²⁵ and R³⁵ and R³⁰ and R⁴⁰ are taken together with the intervening atoms to form a 5- to 6-membered heterocycloalkyl. In some embodiments, R²⁴ and R³⁴, R²⁵ and R³⁵, and R³⁰ and R⁴⁰ are taken together with the intervening atoms to form a 5- to 6-membered heterocycloalkyl.

In some embodiments, each of R³⁷, R³⁹, and R⁴⁰ is selected from methyl and methoxyethyl. In some embodiments, each of R³⁵, R³⁷, and R³⁹ is selected from methyl and methoxyethyl. In some embodiments, each of R³⁵, R³⁷, and R⁴⁰ is selected from methyl and methoxyethyl. In some embodiments, each of R³⁵, R³⁹, and R⁴⁰ is selected from methyl and methoxyethyl. In some embodiments, each of R³⁵, R³⁷, R³⁹, and R⁴⁰ is selected from methyl and methoxyethyl.

In some embodiments, R⁴⁰ is C₂₋₄alkyl optionally substituted with one or more substituents independently selected from halo, —OH, —CH₃, —CF₃, —CHF₂, —OBz, —OCH₃, —SF₅, and —OCHF₂ or R³⁰ and R⁴⁰ are taken together with the intervening atoms to form a 5- to 7-membered heterocycloalkyl. In some embodiments, R⁴⁰ is C₂₋₄alkyl optionally substituted with one or more substituents independently selected from halo, —OH, —CH₃, —CF₃, —CHF₂, —OBz, —OCH₃, and —OCHF₂ or R³⁰ and R⁴⁰ are taken together with the intervening atoms to form a 5- to 7-membered heterocycloalkyl. In some embodiments, R⁴⁰ is C₂₋₄alkyl optionally substituted with one or more substituents independently selected from halo, —OH, —OBz, —OCH₃, —OCF₃, —SF₅, and —OCHF₂ or R³⁰ and R⁴⁰ are taken together with the intervening atoms to form a 5- to 7-membered heterocycloalkyl. In some embodiments, R⁴⁰ is C₂₋₄alkyl optionally substituted with one or more substituents independently selected from halo, —OH, —OBz, —OCH₃, —OCF₃, and —OCHF₂ or R³⁰ and R⁴⁰ are taken together with the intervening atoms to form a 5- to 7-membered heterocycloalkyl. In some embodiments, R⁴⁰ is C₂₋₄alkyl optionally substituted with one or more substituents independently selected from halo, —OH, —CH₃, —CF₃, —CHF₂, —OBz, —OCH₃, —SF₅, and —OCHF₂. In some embodiments, R⁴⁰ is C₂₋₄alkyl optionally substituted with one or more substituents independently selected from halo, —OH, —CH₃, —CF₃, —CHF₂, —OBz, —OCH₃, and —OCHF₂. In some embodiments, R⁴⁰ is C₂₋₄alkyl optionally substituted with one or more substituents independently selected from halo, —OH, —OBz, —OCH₃, —OCF₃, —SF₅, and —OCHF₂. In some embodiments, R⁴⁰ is C₂₋₄alkyl optionally substituted with one or more substituents independently selected from halo, —OH, —OBz, —OCH₃, —OCF₃, and —OCHF₂. In some embodiments, R⁴⁰ is C₂₋₄alkyl optionally substituted with one or more substituents independently selected from halo, —OH, —CH₃, —CF₃, —SF₅, and —OCH₃. In some embodiments, R⁴⁰ is C₂₋₄alkyl optionally substituted with one or more substituents independently selected from halo, —OH, —CH₃, —CF₃, and —OCH₃. In some embodiments, R³⁰ and R⁴⁰ are taken together with the intervening atoms to form a 5- to 7-membered heterocycloalkyl.

In some embodiments, R³⁹ is C₁₋₄alkyl optionally substituted with one or more substituents independently selected from halo, —OH, —CH₃, —CF₃, —CHF₂, —OBz, —OCH₃, —SF₅, and —OCHF₂. In some embodiments, R³⁹ is C₁₋₄alkyl optionally substituted with one or more substituents independently selected from halo, —OH, —CH₃, —CF₃, —CHF₂, —OBz, —OCH₃, and —OCHF₂. In some embodiments, R³⁹ is C₁₋₄alkyl optionally substituted with one or more substituents independently selected from halo, —OH, —OBz, —OCH₃, —OCF₃, —SF₅, and —OCHF₂. In some embodiments, R³⁹ is C₁₋₄alkyl optionally substituted with one or more substituents independently selected from halo, —OH, —OBz, —OCH₃, —OCF₃, and —OCHF₂. In some embodiments, R³⁹ is C₁₋₄alkyl optionally substituted with one or more substituents independently selected from halo, —OH, —CH₃, —CF₃, —SF₅, and —OCH₃. In some embodiments, R³⁹ is C₁₋₄alkyl optionally substituted with one or more substituents independently selected from halo, —OH, —CH₃, —CF₃, and —OCH₃. In some embodiments, R³⁹ is C₁₋₄alkyl optionally substituted with one or more substituents independently selected from —CHF₂, —OBz, —SF₅, and —OCHF₂. In some embodiments, R³⁹ is C₁₋₄alkyl optionally substituted with one or more substituents independently selected from —CHF₂, —OBz, and —OCHF₂.

In some embodiments, R²², R²⁷, and R²⁹ are independently selected from C₁₋₆alkyl. In some embodiments, R²², R²⁷, and R²⁹ are selected from methyl, ethyl, propyl, i-propyl, butyl, i-butyl, and t-butyl. In some embodiments, R²², R²⁷, and R²⁹ are selected from methyl, ethyl, i-propyl, and t-butyl. In some embodiments, R²⁷ is methyl. In some embodiments, R²⁹ is methyl. In some embodiments, R²⁷ and R²⁹ are each methyl.

In some embodiments, R²¹, R²³, R²⁶, and R²⁸ are independently selected from —C₃₋₈carbocycle, −3-10 membered heterocycle, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted. In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted. In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —SF₅, and —OCHF₂. In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, and —OCHF₂. In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted with one or more substituents independently selected from halo, —OH, —OBz, —OCH₃, —OCF₃, and —OCHF₂. In some embodiments, R²¹, R²³, R²⁶, and R²⁸ are independently selected from —CH₂—(C₃₋₈carbocycle), and —CH₂-(3-10 membered heterocycle). In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —SF₅, and —OCH₃. In some embodiments, R²¹, R²³, R²⁶, and R²⁸ are independently selected from —(C₁₋₄alkylene)-(C₃₋₈carbocycle) and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, and —OCH₃. In some embodiments, R²¹, R²³, R²⁶, and R²⁸ are independently selected from phenylmethyl and pyridinylmethyl, wherein the phenyl and pyridinyl are optionally substituted with one or more substituents independently selected from halo, —OH, —CH₃, —CF₃, —SF₅, and —OCH₃. In some embodiments, R²¹, R²³, R²⁶, and R²⁸ are independently selected from phenylmethyl and pyridinylmethyl, wherein the phenyl and pyridinyl are optionally substituted with one or more substituents independently selected from halo, —OH, —CH₃, —CF₃, and —OCH₃. In some embodiments R²¹, R²³, R²⁶ and R²⁸ are independently selected from:

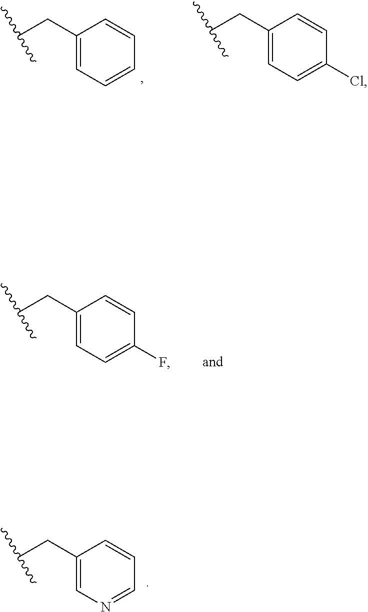 [SMILES: *Cc1ccccc1 ; *Cc1ccc(Cl)cc1 ; *Cc1ccc(F)cc1 ; *Cc1cccnc1]

In some embodiments, R²¹ is

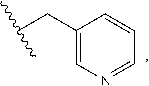 [SMILES: *Cc1cccnc1]

and R²³, R²⁶, and R²⁸ are independently selected from

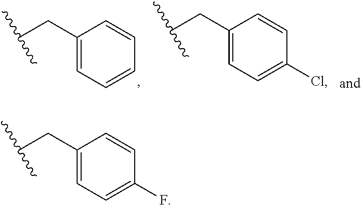

In some embodiments, R²¹ is

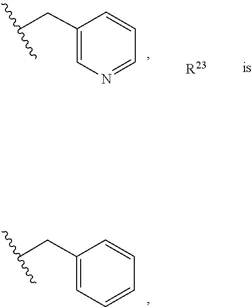

and R²⁶ and R²⁸ are independently selected from

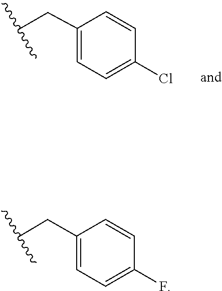 [SMILES: *Cc1ccc(Cl)cc1 ; *Cc1ccc(F)cc1]

In some embodiments, R²¹ is

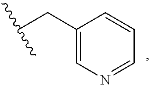 [SMILES: *Cc1cccnc1]

### R²³ is

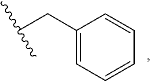 [SMILES: *Cc1ccccc1]

### R²⁶ is

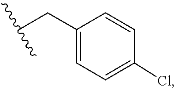 [SMILES: *Cc1ccc(Cl)cc1]

### and R²⁸ is

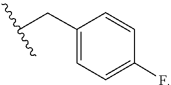 [SMILES: *Cc1ccc(F)cc1]

In some embodiments, the cyclic peptide is represented by Formula IIa:

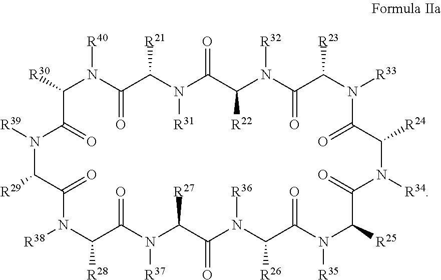

In some embodiments, the cyclic peptide is represented by Formula IIb:

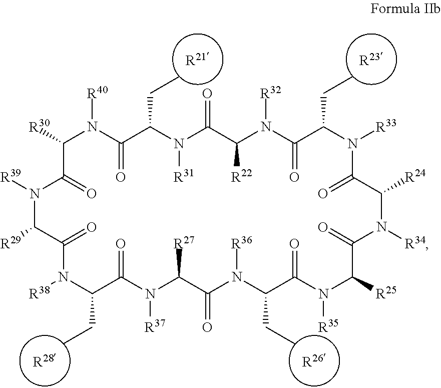

wherein R^{21′}, R^{23′}, R^{26′} and R^{28′} are independently selected from optionally substituted phenyl and optionally substituted 5- or 6-membered heteroaryl.

In some embodiments, R^{21′}, R^{23′}, R^{26′}, and R^{28′} are independently selected from:

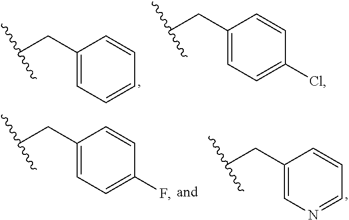

In some embodiments, R^{21′} is

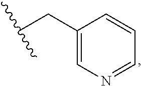 [SMILES: *Cc1cccnc1]

and R^{23′} R^{26′} and R^{28′} are independently selected from

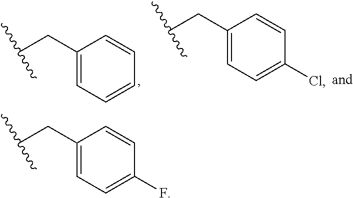

In some embodiments, R^{21′} is

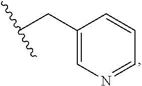 [SMILES: *Cc1cccnc1]

### R^{23′} is

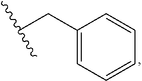 [SMILES: *Cc1ccccc1]

and R^{26′} and R^{28′} are independently selected from

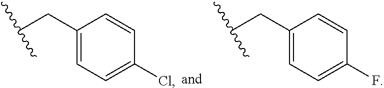 [SMILES: *Cc1ccc(Cl)cc1 ; *Cc1ccc(F)cc1]

In some embodiments, R^{21′} is

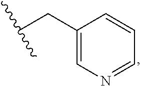 [SMILES: *Cc1cccnc1]

### R^{23′} is

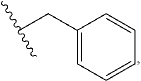 [SMILES: *Cc1ccccc1]

### R^{26′} is

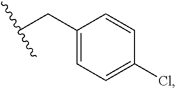 [SMILES: *Cc1ccc(Cl)cc1]

### and R^{28′} is

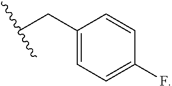 [SMILES: *Cc1ccc(F)cc1]

In some embodiments, the cyclic peptide is represented by Formula IIc:

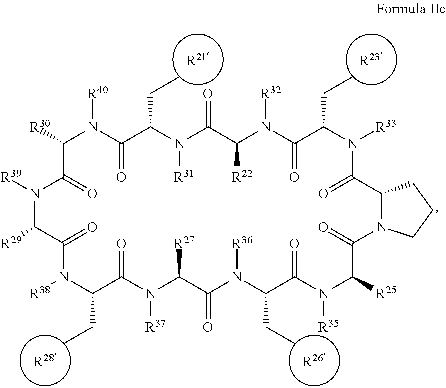

wherein R^{21′}, R^{23′}, R^{26′}, and R²⁸ are independently selected from optionally substituted phenyl and optionally substituted 5- or 6-membered heteroaryl.

In some embodiments, the cyclic peptide is represented by Formula IId:

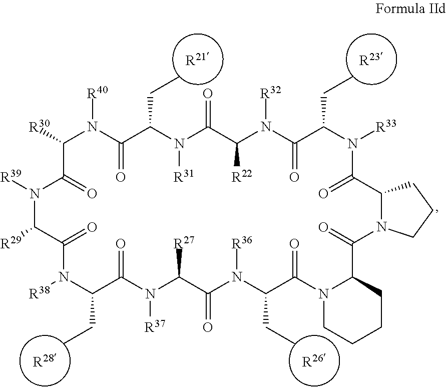

wherein R^{21′}, R^{23′}, R^{26′}, and R²⁸ are independently selected from optionally substituted phenyl and optionally substituted 5- or 6-membered heteroaryl.

In some embodiments, the cyclic peptide is represented by Formula IIe:

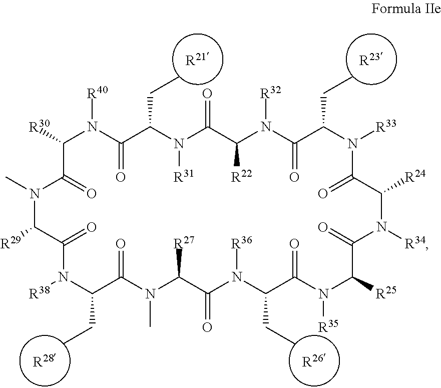

wherein R^{21′}, R^{23′}, R^{26′}, and R^{28′} are independently selected from optionally substituted phenyl and optionally substituted 5- or 6-membered heteroaryl.

In some embodiments, the cyclic peptide is represented by Formula IIf:

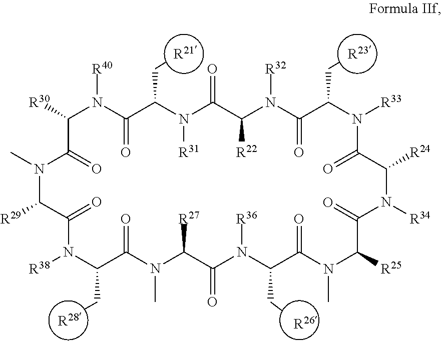

wherein R^{21′}, R^{23′}, R^{26′}, and R^{28′} are independently selected from optionally substituted phenyl and optionally substituted 5- or 6-membered heteroaryl.

In some embodiments, the cyclic peptide is represented by Formula IIg:

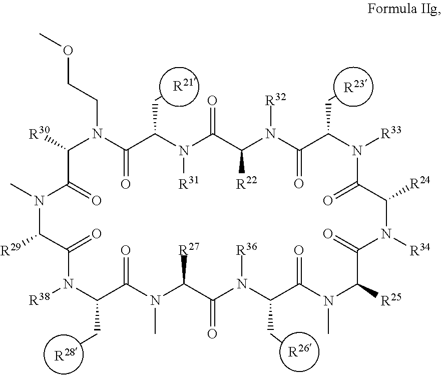

wherein R^{21′}, R^{23′}, R^{26′}, and R^{28′} are independently selected from optionally substituted phenyl and optionally substituted 5- or 6-membered heteroaryl.

In some embodiments, the cyclic peptide is represented by Formula III:

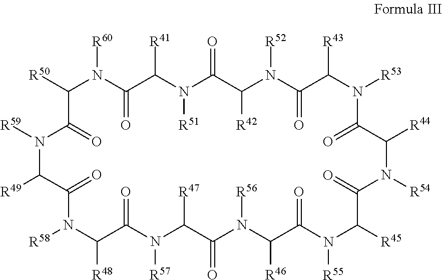

wherein:

R⁴¹, R⁴⁵, R⁴⁶, and R⁴⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂;

R⁴² is selected from hydrogen and C₁₋₆alkyl;

R⁴³ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁴⁴ is hydrogen or C₁₋₄alkyl, or R⁴⁴ and R⁵⁴ are taken together with the intervening atoms to form a 4- to 7-membered heterocycloalkyl;

R⁴⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, —SF₅, and —OCHF₂, or R⁴⁷ and R⁵⁷ are taken together with the intervening atoms to form a 4- to 7-membered heterocycloalkyl;

R⁴⁹ is hydrogen or C₁₋₆alkyl, or R⁴⁹ and R⁵⁹ are taken together with the intervening atoms to form a 4- to 7-membered heterocycloalkyl;

R⁵⁰ is hydrogen or C₁₋₄alkyl, or R⁵⁰ and R⁶⁰ are taken together with the intervening atoms to form a 4- to 7-membered heterocycloalkyl;

R⁵¹, R⁵³, R⁵⁶, and R⁵⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁵² and R⁵⁵ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂;

R⁵⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁴ and R⁴⁴ are taken together with the intervening atoms to form a 4- to 7-membered heterocycloalkyl;

R⁵⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁷ and R⁴⁷ are taken together with the intervening atoms to form a 4- to 7-membered heterocycloalkyl;

R⁵⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁹ and R⁴⁹ are taken together with the intervening atoms to form a 4- to 7-membered heterocycloalkyl;

R⁶⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁶⁰ and R⁵⁰ are taken together with the intervening atoms to form a 4- to 7-membered heterocycloalkyl.

In some embodiments, the cyclic peptide is represented by Formula III:

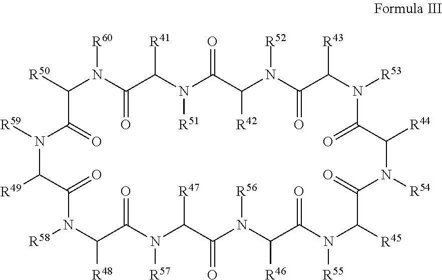

wherein:

R⁴¹, R⁴⁵, R⁴⁶, and R⁴⁸ are independently selected from hydrogen, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂;

R⁴² is selected from hydrogen and C₁₋₆alkyl;

R⁴³ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁴⁴ is hydrogen or C₁₋₄alkyl, or R⁴⁴ and R⁵⁴ are taken together with the intervening atoms to form a 4- to 7-membered heterocycloalkyl;

R⁴⁷ is selected from hydrogen; and C₁₋₆alkyl optionally substituted with one or more substituents independently selected from halo, —SCH₃, —SOCH₃, —SO₂CH₃, —OH, —CN, —NO₂, C₁₋₄alkyl, —OH, —OBz, —OCH₃, —OCF₃, and —OCHF₂, or R⁴⁷ and R⁵⁷ are taken together with the intervening atoms to form a 4- to 7-membered heterocycloalkyl;

R⁴⁹ is hydrogen or C₁₋₆alkyl, or R⁴⁹ and R⁵⁹ are taken together with the intervening atoms to form a 4- to 7-membered heterocycloalkyl;

R⁵⁰ is hydrogen or C₁₋₄alkyl, or R⁵⁰ and R⁶⁰ are taken together with the intervening atoms to form a 4- to 7-membered heterocycloalkyl;

R⁵¹, R⁵³, R⁵⁶, and R⁵⁸ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁵² and R⁵⁵ are independently selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂;

R⁵⁴ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁴ and R⁴⁴ are taken together with the intervening atoms to form a 4- to 7-membered heterocycloalkyl;

R⁵⁷ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁷ and R⁴⁷ are taken together with the intervening atoms to form a 4- to 7-membered heterocycloalkyl;

R⁵⁹ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁹ and R⁴⁹ are taken together with the intervening atoms to form a 4- to 7-membered heterocycloalkyl;

R⁶⁰ is selected from hydrogen; and C₁₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁶⁰ and R⁵⁰ are taken together with the intervening atoms to form a 4- to 7-membered heterocycloalkyl.

In some embodiments, R⁵¹ is hydrogen. In some embodiments, R⁵³ is hydrogen. In some embodiments, R⁵⁶ is hydrogen. In some embodiments, R⁵⁸ is hydrogen. In some embodiments, R⁵¹ and R⁵³ are each hydrogen. In some embodiments, R⁵¹ and R⁵⁶ are each hydrogen. In some embodiments, R⁵¹ and R⁵⁸ are each hydrogen. In some embodiments, R⁵³ and R⁵⁶ are each hydrogen. In some embodiments, R⁵³ and R⁵⁸ are each hydrogen. In some embodiments, R⁵⁶ and R⁵⁸ are each hydrogen. In some embodiments, R⁵¹, R⁵³, and R⁵⁶ are each hydrogen. In some embodiments, R⁵¹, R⁵³, and R⁵⁸ are each hydrogen. In some embodiments, R⁵¹, R⁵⁶, and R⁵⁸ are each hydrogen. In some embodiments, R⁵³, R⁵⁶, and R⁵⁸ are each hydrogen. In some embodiments, R⁵¹, R⁵³, R⁵⁶, and R⁵⁸ are each hydrogen.

In some embodiments, at least four of R⁵², R⁵⁴, R⁵⁵, R⁵⁷, R⁵⁹, and R⁶⁰ are not hydrogen. In some embodiments, four of R⁵², R⁵⁴, R⁵⁵, R⁷, R⁵⁹, and R⁶⁰ are not hydrogen. In some embodiments, R⁵¹, R⁵⁴, R⁵⁵, and R⁵⁷ are not hydrogen. In some embodiments, R⁵², R⁵⁴, R⁵⁵, and R⁵⁹ are not hydrogen. In some embodiments, R⁵², R⁵⁴, R⁵⁵, and R⁶⁰ are not hydrogen. In some embodiments, R⁵¹, R⁵⁴, R⁵⁷, and R⁵⁹ are not hydrogen. In some embodiments, R⁵², R⁵⁴, R⁵⁷, and R⁶⁰ are not hydrogen. In some embodiments, R⁵², R⁵⁴, R⁵⁹, and R⁶⁰ are not hydrogen. In some embodiments, R⁵², R⁵⁵, R⁵⁷, and R⁵⁹ are not hydrogen. In some embodiments, R⁵², R⁵⁵, R⁵⁷, and R⁶⁰ are not hydrogen. In some embodiments, R⁵², R⁵⁵, R⁵⁹, and R⁶⁰ are not hydrogen. In some embodiments, R⁵², R⁵⁷, R⁵⁹, and R⁶⁰ are not hydrogen. In some embodiments, R⁵⁴, R⁵⁵, R⁵⁷, and R⁵⁹ are not hydrogen. In some embodiments, R⁵⁴, R⁵⁵, R⁵⁷, and R⁶⁰ are not hydrogen. In some embodiments, R⁵⁴, R⁵⁵, R⁵⁹, and R⁶⁰ are not hydrogen. In some embodiments, R⁵⁴, R⁷, R⁵⁹, and R⁶⁰ are not hydrogen. In some embodiments, R⁵⁵, R⁵⁷, R⁵⁹, and R⁶⁰ are not hydrogen. In some embodiments, at least five of R⁵², R⁵⁴, R⁵⁵, R⁵⁷, R⁵⁹, and R⁶⁰ are not hydrogen. In some embodiments, five of R⁵², R⁵⁴, R⁵⁵, R⁵⁷, R⁵⁹, and R⁶⁰ are not hydrogen. In some embodiments, R⁵², R⁵⁴, R⁵⁵, R⁵⁷, and R⁵⁹ are not hydrogen. In some embodiments, R⁵², R⁵⁴, R⁵⁵, R⁵⁷, and R⁶⁰ are not hydrogen. In some embodiments, R⁵², R⁵⁵, R⁵⁷, R⁵⁹, and R⁶⁰ are not hydrogen. In some embodiments, R⁵⁴, R⁵⁵, R⁵⁷, R⁵⁹, and R⁶⁰ are not hydrogen. In some embodiments, R⁵², R⁵⁴, R⁵⁵, R⁵⁷, R⁵⁹, and R⁶⁰ are not hydrogen.

In some embodiments, at least one of R⁴⁴ and R⁵⁴, R⁴⁷ and R⁵⁷, R⁴⁹ and R⁵⁹, and R⁵⁰ and R⁶⁰ are taken together with the intervening atoms to form a 4- to 7-membered heterocycloalkyl. In some embodiments, at least two of R⁴⁴ and R⁵⁴, R⁴⁷ and R⁵⁷, R⁴⁹ and R⁵⁹, and R⁵⁰ and R⁶⁰ are taken together with the intervening atoms to form a 4- to 7-membered heterocycloalkyl. In some embodiments, R⁴⁴ and R⁵⁴ are taken together with the intervening atoms to form a 4- to 7-membered heterocycloalkyl. In some embodiments, R⁴⁷ and R⁵⁷ are taken together with the intervening atoms to form a 4- to 7-membered heterocycloalkyl. In some embodiments, R⁴⁹ and R⁵⁹ are taken together with the intervening atoms to form a 4- to 7-membered heterocycloalkyl. In some embodiments, R⁵⁰ and R⁶⁰ are taken together with the intervening atoms to form a 4- to 7-membered heterocycloalkyl. In some embodiments, R⁴⁴ and R⁵⁴ and R⁴⁷ and R⁵⁷ are taken together with the intervening atoms to form a 4- to 7-membered heterocycloalkyl. In some embodiments, R⁴⁴ and R⁵⁴ and R⁴⁹ and R⁵⁹ are taken together with the intervening atoms to form a 4- to 7-membered heterocycloalkyl. In some embodiments, R⁴⁴ and R⁵⁴ and R⁵⁰ and R⁶⁰ are taken together with the intervening atoms to form a 4- to 7-membered heterocycloalkyl. In some embodiments, R⁴⁷ and R⁵⁷ and R⁴⁹ and R⁵⁹ are taken together with the intervening atoms to form a 4- to 7-membered heterocycloalkyl. In some embodiments, R⁴⁷ and R⁵⁷ and R⁵⁰ and R⁶⁰ are taken together with the intervening atoms to form a 4- to 7-membered heterocycloalkyl. In some embodiments, R⁴⁹ and R⁵⁹ and R⁵⁰ and R⁶⁰ are taken together with the intervening atoms to form a 4- to 7-membered heterocycloalkyl. In some embodiments, R⁴⁴ and R⁵⁴, R⁴⁷ and R⁵⁷, and R⁴⁹ and R⁵⁹ are taken together with the intervening atoms to form a 4- to 7-membered heterocycloalkyl. In some embodiments, R⁴⁴ and R⁵⁴, R⁴⁷ and R⁵⁷, and R⁵⁰ and R⁶⁰ are taken together with the intervening atoms to form a 4- to 7-membered heterocycloalkyl. In some embodiments, R⁴⁴ and R⁵⁴, R⁴⁹ and R⁵⁹, and R⁵⁰ and R⁶⁰ are taken together with the intervening atoms to form a 4- to 7-membered heterocycloalkyl. In some embodiments, R⁴⁷ and R⁵⁷, R⁴⁹ and R⁵⁹, and R⁵⁰ and R⁶⁰ are taken together with the intervening atoms to form a 4- to 7-membered heterocycloalkyl. In some embodiments, R⁴⁴ and R⁵⁴, R⁴⁷ and R⁵⁷, R⁴⁹ and R⁵⁹, and R⁵⁰ and R⁶⁰ are taken together with the intervening atoms to form a 4- to 7-membered heterocycloalkyl.

In some embodiments, each of R⁵², R⁵⁵, and R⁵⁹ is selected from methyl, ethyl, and methoxyethyl. In some embodiments, each of R⁵², R⁵⁵, and R⁶⁰ is selected from methyl, ethyl, and methoxyethyl. In some embodiments, each of R⁵², R⁵⁹, and R⁶⁰ is selected from methyl, ethyl, and methoxyethyl. In some embodiments, each of R⁵², R⁵⁹, and R⁶⁰ is selected from methyl, ethyl, and methoxyethyl. In some embodiments, each of R⁵², R⁵⁵, R⁵⁹, and R⁶⁰ is selected from methyl, ethyl, and methoxyethyl. In some embodiments, each of R⁵², R⁵⁵, R⁵⁷, R⁵⁹, and R⁶⁰ is selected from methyl, ethyl, and methoxyethyl.

In some embodiments, R⁶⁰ is C₂₋₄alkyl optionally substituted with one or more substituents independently selected from halo, —SCH₃, —SOCH₃, —SO₂CH₃, —CF₃, —CHF₂, —CN, —NO₂, C₁₋₄alkyl, —OH, —OBz, —OCH₃, —OCF₃, —SF₅, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CF₃, —CHF₂, —CN, —NO₂, C₁₋₄alkyl, —OH, —OBz, —OCH₃, —OCF₃, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —OH, —CH₃, —CF₃, —CHF₂, —OBz, —OCH₃, —SF₅, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —OH, —CH₃, —CF₃, —CHF₂, —OBz, —OCH₃, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —OH, —OBz, —OCH₃, —OCF₃, —SF₅, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —OH, —OBz, —OCH₃, —OCF₃, and —OCHF₂, or R⁵⁰ and R⁶⁰ are taken together with the intervening atoms to form a 4- to 7-membered heterocycloalkyl. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂. In some embodiments, R⁶⁰ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂. In some embodiments, R⁶⁰ is C₂₋₄alkyl optionally substituted with one or more substituents independently selected from halo, —OH, —CH₃, —CF₃, —CHF₂, —OBz, —OCH₃, —SF₅, and —OCHF₂. In some embodiments, R⁶⁰ is C₂₋₄alkyl optionally substituted with one or more substituents independently selected from halo, —OH, —CH₃, —CF₃, —CHF₂, —OBz, —OCH₃, and —OCHF₂. In some embodiments, R⁶⁰ is C₂₋₄alkyl optionally substituted with one or more substituents independently selected from halo, —OH, —OBz, —OCH₃, —OCF₃, —SF₅, and —OCHF₂. In some embodiments, R⁶⁰ is C₂₋₄alkyl optionally substituted with one or more substituents independently selected from halo, —OH, —OBz, —OCH₃, —OCF₃, and —OCHF₂. In some embodiments, R⁶⁰ is C₂₋₄alkyl optionally substituted with one or more substituents independently selected from halo, —OH, —CH₃, —CF₃, —SF₅, and —OCH₃. In some embodiments, R⁶⁰ is C₂₋₄alkyl optionally substituted with one or more substituents independently selected from halo, —OH, —CH₃, —CF₃, and —OCH₃. In some embodiments, R⁵⁰ and R⁶⁰ are taken together with the intervening atoms to form a 4- to 7-membered heterocycloalkyl.

In some embodiments, R⁵⁹ is C₂₋₄alkyl optionally substituted with one or more substituents independently selected from halo, —CF₃, —CHF₂, —SCH₃, —SOCH₃, —SO₂CH₃, —CN, —NO₂, C₁₋₄alkyl, —OH, —OBz, —OCH₃, —OCF₃, —SF₅, and —OCHF₂. In some embodiments, R⁵⁹ is C₂-4alkyl optionally substituted with one or more substituents independently selected from halo, —CF₃, —CHF₂, —SCH₃, —SOCH₃, —SO₂CH₃, —CN, —NO₂, C₁₋₄alkyl, —OH, —OBz, —OCH₃, —OCF₃, and —OCHF₂. In some embodiments, R⁵⁹ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, —SF₅, and —OCHF₂. In some embodiments, R⁵⁹ is C₂₋₄alkyl optionally substituted with one or more substituents independently selected from halo, —SCH₃, —SOCH₃, —SO₂CH₃, —CN, —NO₂, C₁₋₄alkyl, —OH, —OBz, —OCH₃, —OCF₃, and —OCHF₂. In some embodiments, R⁵⁹ is C₂₋₄alkyl optionally substituted with one or more substituents independently selected from halo, —SCH₃, —SOCH₃, —SO₂CH₃, —CN, —NO₂, —CH₃, —OH, —OBz, —OCH₃, —OCF₃, —SF₅, and —OCHF₂. In some embodiments, R⁵⁹ is C₂₋₄alkyl optionally substituted with one or more substituents independently selected from halo, —SCH₃, —SOCH₃, —SO₂CH₃, —CN, —NO₂, —CH₃, —OH, —OBz, —OCH₃, —OCF₃, and —OCHF₂. In some embodiments, R³⁹ is C₂₋₄alkyl optionally substituted with one or more substituents independently selected from halo, —OH, —CH₃, —CF₃, —CHF₂, —OBz, —OCH₃, —SF₅, and —OCHF₂. In some embodiments, R³⁹ is C₂₋₄alkyl optionally substituted with one or more substituents independently selected from halo, —OH, —CH₃, —CF₃, —CHF₂, —OBz, —OCH₃, and —OCHF₂. In some embodiments, R³⁹ is C₂₋₄alkyl optionally substituted with one or more substituents independently selected from halo, —OH, —OBz, —OCH₃, —OCF₃, —SF₅, and —OCHF₂. In some embodiments, R³⁹ is C₂₋₄alkyl optionally substituted with one or more substituents independently selected from halo, —OH, —OBz, —OCH₃, —OCF₃, and —OCHF₂. In some embodiments, R³⁹ is C₂₋₄alkyl optionally substituted with one or more substituents independently selected from halo, —OH, —CH₃, —CF₃, —SF₅, and —OCH₃. In some embodiments, R³⁹ is C₂₋₄alkyl optionally substituted with one or more substituents independently selected from halo, —OH, —CH₃, —CF₃, and —OCH₃. In some embodiments, R³⁹ is C₂₋₄alkyl optionally substituted with one or more substituents independently selected from —CHF₂, —OBz, and —OCHF₂.

In some embodiments, R⁴², R⁴⁷, and R⁴⁹ are independently selected from C₁₋₆alkyl. In some embodiments, R⁴², R⁴⁷, and R⁴⁹ are selected from methyl, ethyl, propyl, i-propyl, butyl, i-butyl, and t-butyl. In some embodiments, R⁴², R⁴⁷, and R⁴⁹ are selected from methyl, ethyl, i-propyl, and t-butyl. In some embodiments, R⁴² is methyl. In some embodiments, R⁴⁷ is methyl, ethyl, i-propyl, or t-butyl. In some embodiments, R⁴⁹ is methyl. In some embodiments, R⁴² is methyl, R⁴⁷ is methyl, ethyl, i-propyl, or t-butyl, and R⁴⁹ is hydrogen. In some embodiments, R⁴² is methyl, R⁴⁷ is methyl, and R⁴⁹ is hydrogen. In some embodiments, R⁴² is methyl, R⁴⁷ is ethyl, and R⁴⁹ is hydrogen. In some embodiments, R⁴² is methyl, R⁴⁷ is i-propyl, and R⁴⁹ is hydrogen. In some embodiments, R⁴² is methyl, R⁴⁷ is t-butyl, and R⁴⁹ is hydrogen. In some embodiments, R⁴² is methyl, R⁴⁷ is methyl, ethyl, i-propyl, or t-butyl, and R⁴⁹ is methyl. In some embodiments, R⁴² is methyl, R⁴⁷ is methyl, and R⁴⁹ is methyl. In some embodiments, R⁴² is methyl, R⁴⁷ is ethyl, and R⁴⁹ is methyl. In some embodiments, R⁴² is methyl, R⁴⁷ is i-propyl, and R⁴⁹ is methyl. In some embodiments, R⁴² is methyl, R⁴⁷ is t-butyl, and R⁴⁹ is methyl.

In some embodiments, R⁴¹, R⁴⁵, R⁴⁶, and R⁴⁸ are independently selected from —C₃₋₈carbocycle, −3-10 membered heterocycle, —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, —SF₅, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —SCH₃, —SOCH₃, —SO₂CH₃, —CN, —NO₂, C₁₋₄alkyl, —OH, —CH₃, —CF₃, —CHF₂, —OBz, —OCH₃, —OCF₃, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, —SF₅, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —CHF₂, —OBz, —OCH₃, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —OBz, —OCH₃, —OCF₃, —SF₅, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —OBz, —OCH₃, —OCF₃, and —OCHF₂. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, —SF₅, and —OCH₃. In some embodiments, R⁴¹, R⁴⁵, R⁴⁶, and R⁴⁸ are independently selected from —(C₁₋₄alkylene)-(C₃₋₈carbocycle), and —(C₁₋₄alkylene)-(3-10 membered heterocycle), wherein the C₃₋₈carbocycle and 3-10 membered heterocycle are optionally substituted with one or more substituents independently selected from halo, —OH, —CH₃, —CF₃, and —OCH₃. In some embodiments, R⁴¹, R⁴⁵, R⁴⁶, and R⁴⁸ are independently selected from —CH₂—(C₃₋₈carbocycle), and —CH₂-(3-10 membered heterocycle). In some embodiments, R⁴¹, R⁴⁵, R⁴⁶, and R⁴⁸ are independently selected from phenylmethyl, pyridinylmethyl, and thiazolylmethyl, wherein the phenyl, pyridinyl, and thiazolyl are optionally substituted with one or more substituents independently selected from halo, —OH, —CH₃, —CF₃, and —OCH₃. In some embodiments. R^{41′} R^{45′}. R^{46′}, and R^{48′} are independently selected from:

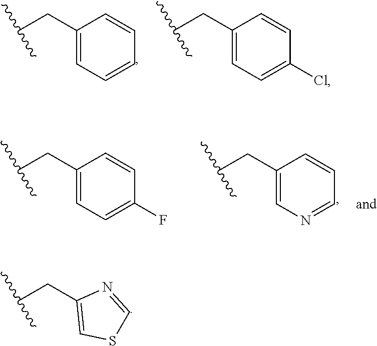

In some embodiments, R^{41′} is

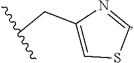 [SMILES: *Cc1cscn1]

and R^{45′}, R^{46′}, and R^{48′} are independently selected from

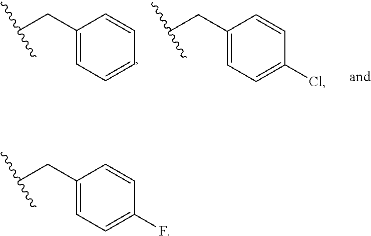

In some embodiments, R^{41′} is

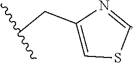 [SMILES: *Cc1cscn1]

### R^{45′} is

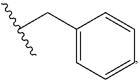 [SMILES: *Cc1ccccc1]

and R^{46′} and R^{48′} are independently selected from

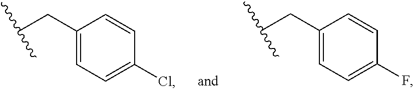 [SMILES: *Cc1ccc(Cl)cc1 ; *Cc1ccc(F)cc1]

In some embodiments, R^{41′} is

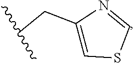 [SMILES: *Cc1cscn1]

In some embodiments R^{41′} is

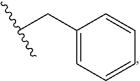 [SMILES: *Cc1ccccc1]

### R^{45′} is

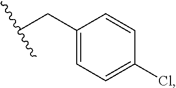 [SMILES: *Cc1ccc(Cl)cc1]

### and R^{48′} is

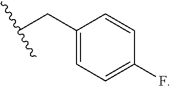 [SMILES: *Cc1ccc(F)cc1]

In some embodiments, R^{41′} is

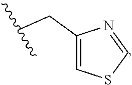 [SMILES: *Cc1cscn1]

### R^{45′} is

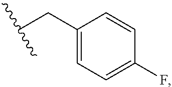 [SMILES: *Cc1ccc(F)cc1]

and R^{46′} and R^{48′} are independently selected from

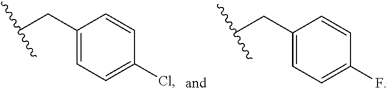 [SMILES: *Cc1ccc(Cl)cc1 ; *Cc1ccc(F)cc1]

In some embodiments, R^{41′} is

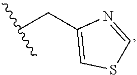 [SMILES: *Cc1cscn1]

### R^{45′} is

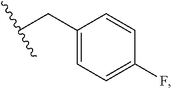 [SMILES: *Cc1ccc(F)cc1]

### R^{46′} is

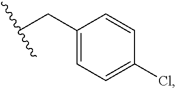 [SMILES: *Cc1ccc(Cl)cc1]

### and R^{48′} is

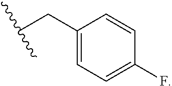 [SMILES: *Cc1ccc(F)cc1]

In some embodiments, the cyclic peptide is represented by Formula IIIa:

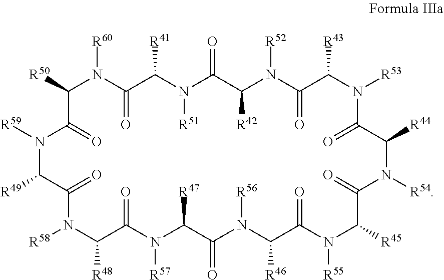

In some embodiments, the cyclic peptide is represented by Formula IIb:

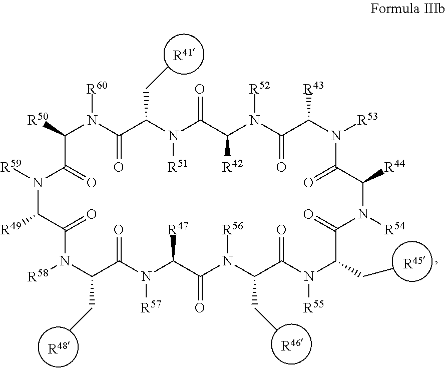

wherein R^{41′}, R^{45′}, R^{46′} and R^{48′} are independently selected from optionally substituted phenyl and optionally substituted 5- or 6-membered heteroaryl.

In some embodiments, R⁴¹, R^{45′}, R^{46′}, and R^{48′} are independently selected from:

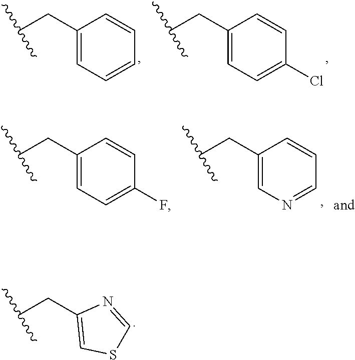

In some embodiments R^{41′} is

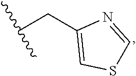 [SMILES: *Cc1cscn1]

and R^{45′}, R^{46′}, and R^{48′} are independently selected from

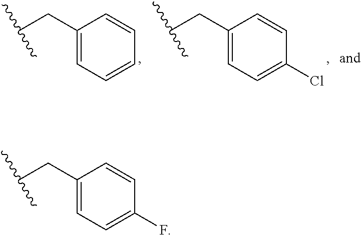

In some embodiments, R^{41′} is

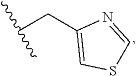 [SMILES: *Cc1cscn1]

### and R^{45′} is

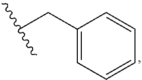 [SMILES: *Cc1ccccc1]

and R^{46′} and R^{48′} are independently selected from

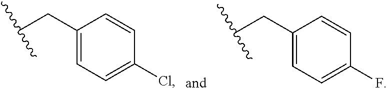 [SMILES: *Cc1ccc(Cl)cc1 ; *Cc1ccc(F)cc1]

In some embodiments, R^{41′} is

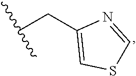 [SMILES: *Cc1cscn1]

### R^{45′} is

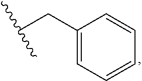 [SMILES: *Cc1ccccc1]

### R^{46′} is

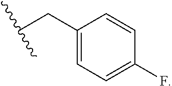 [SMILES: *Cc1ccc(F)cc1]

### and R^{48′} is

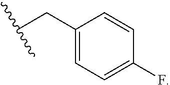 [SMILES: *Cc1ccc(F)cc1]

In some embodiments R^{41′} is

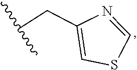 [SMILES: *Cc1cscn1]

### R^{45′} is

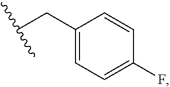 [SMILES: *Cc1ccc(F)cc1]

and R^{46′} and R^{48′} are independently selected from

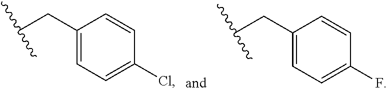 [SMILES: *Cc1ccc(Cl)cc1 ; *Cc1ccc(F)cc1]

In some embodiments, R^{41′} is

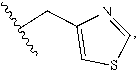 [SMILES: *Cc1cscn1]

### R⁴⁵ is

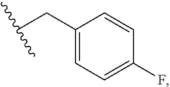 [SMILES: *Cc1ccc(F)cc1]

### R^{46′} is

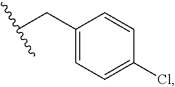 [SMILES: *Cc1ccc(Cl)cc1]

### and R^{48′} is

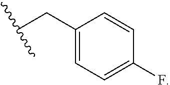 [SMILES: *Cc1ccc(F)cc1]

In some embodiments, the cyclic peptide is represented by Formula IIIc:

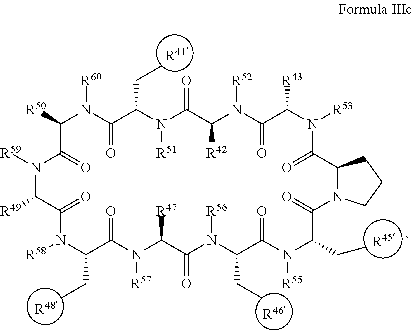

wherein R^{41′}, R^{45′}, R^{46′}, and R^{48′} are independently selected from optionally substituted phenyl and optionally substituted 5- or 6-membered heteroaryl.

In some embodiments, the cyclic peptide is represented by Formula IIId:

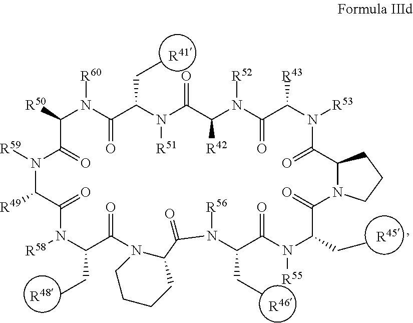

wherein R^{41′}, R^{45′}, R^{46′}, and R^{48′} are independently selected from optionally substituted phenyl and optionally substituted 5- or 6-membered heteroaryl.

In some embodiments, the cyclic peptide is represented by Formula IIIe:

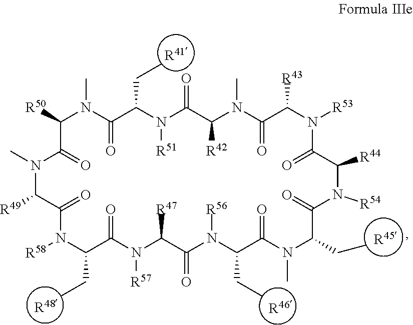

wherein R^{41′}, R^{45′}, R^{46′}, and R^{48′} are independently selected from optionally substituted phenyl and optionally substituted 5- or 6-membered heteroaryl.

In some embodiments, the cyclic peptide is represented by Formula IIIf:

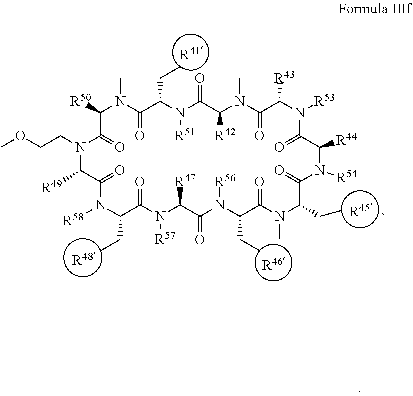

wherein R^{41′}, R^{45′}, R^{46′}, and R^{48′} are independently selected from optionally substituted phenyl and optionally substituted 5- or 6-membered heteroaryl.

In some embodiments, the cyclic peptide is represented by Formula IIIg:

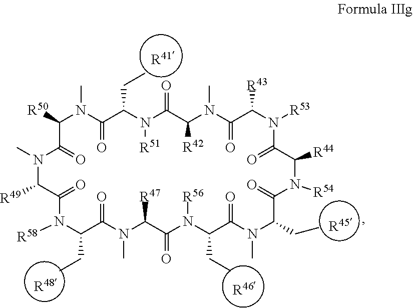

wherein R^{41′}, R^{45′}, R^{46′}, and R^{48′} are independently selected from optionally substituted phenyl and optionally substituted 5- or 6-membered heteroaryl.

In some embodiments, the cyclic peptide is represented by Formula IIIh:

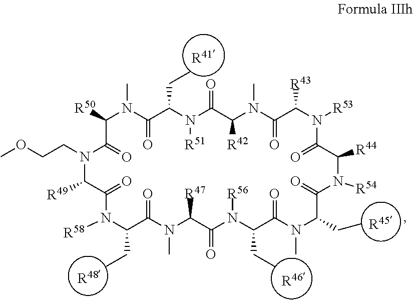

wherein R^{41′}, R^{45′}, R^{46′}, and R^{48′} are independently selected from optionally substituted phenyl and optionally substituted 5- or 6-membered heteroaryl.

In some embodiments, the cyclic peptide is selected from those in Table 3 and Table 4, or a pharmaceutically acceptable salt of any one thereof.

In some embodiments, the cyclic peptides disclosed herein possess a cellular permeability value greater than 1.0×10⁻⁷ cm s⁻¹. In some embodiments, the cyclic peptides disclosed herein possess a cellular permeability value greater than 1.0×10⁻⁶ cm s⁻¹. In some embodiments, the cyclic peptides disclosed herein possess a cellular permeability value greater than 1.0×10⁻⁵ cm s⁻¹. In some embodiments, the cyclic peptides disclosed herein possess a cellular permeability value greater than 1.0×10⁻⁴ cm s⁻¹. In some embodiments, the cyclic peptides disclosed herein possess a cellular permeability value greater than 1.0×10⁻³ cm s⁻¹. In some embodiments, the cyclic peptides disclosed herein possess a cellular permeability value greater than 0.01 cm s⁻¹. In some embodiments, the cyclic peptides disclosed herein possess a cellular permeability value greater than 0.1 cm s⁻¹. In some embodiments, the cyclic peptides disclosed herein possess a cellular permeability value greater than 1.0 cm s⁻¹. In some embodiments, the cellular permeability value of the cyclic peptides disclosed herein is determined by a Caco-2 assay. In some embodiments, the cellular permeability value of the cyclic peptides disclosed herein is determined by a MDR1-MDCK assay.

In some embodiments, the cyclic peptides disclosed herein possess a solubility value greater than 5.0×10⁻⁸ M. In some embodiments, the cyclic peptides disclosed herein possess a solubility value greater than 5.0×10⁻⁷ M. In some embodiments, the cyclic peptides disclosed herein possess a solubility value greater than 5.0×10⁻⁶ M. In some embodiments, the cyclic peptides disclosed herein possess a solubility value greater than 5.0×10⁻⁵ M. In some embodiments, the cyclic peptides disclosed herein possess a solubility value greater than 5.0×10⁻⁴ M. In some embodiments, the cyclic peptides disclosed herein possess a solubility value greater than 5.0×10⁻³ M. In some embodiments, the cyclic peptides disclosed herein possess a solubility value greater than 0.05 M. In some embodiments, the cyclic peptides disclosed herein possess a solubility value greater than 0.5 M. In some embodiments, the cyclic peptides disclosed herein possess a solubility value greater than 5.0 M. In some embodiments, the solubility value of the cyclic peptides disclosed herein is determined by a kinetic solubility assay. In some embodiments, the solubility value of the cyclic peptides disclosed herein is determined by an equilibrium solubility assay. In some embodiments, the solubility value of the cyclic peptides disclosed herein is determined by a nephelometric assay. In some embodiments, the solubility value of the cyclic peptides disclosed herein is determined by a turbidimetric assay. In some embodiments, the solubility value of the cyclic peptides disclosed herein is determined by a direct UV assay.

The compounds disclosed herein, in some embodiments, are used in different enriched isotopic forms, e.g., enriched in the content of ²H, ³H, ¹¹C, ¹³C and/or ¹⁴C. Deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the cyclic peptides disclosed herein, whether radioactive or not, are encompassed within the scope of this disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

The cyclic peptides disclosed herein also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

### Pharmaceutical Formulations

A cyclic peptide of the present disclosure is formulated in any suitable pharmaceutical formulation. A pharmaceutical formulation of the present disclosure typically contains an active ingredient (e.g., a cyclic peptide disclosed herein), and one or more pharmaceutically acceptable excipients or carriers, including but not limited to: inert solid diluents and fillers, diluents, sterile aqueous solution and various organic solvents, permeation enhancers, solubilizers, and adjuvants. In some embodiments, the pharmaceutical acceptable carriers or excipients are selected from water, alcohol, glycerol, chitosan, alginate, chondroitin, Vitamin E, mineral oil, and dimethyl sulfoxide (DMSO).

Pharmaceutical formulations are provided in any suitable form, which is determined based on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of each cyclic peptide administered will be dependent on the mammal being treated, the severity of the disorder or condition, the rate of administration, the disposition of the cyclic peptide and the discretion of the prescribing physician In some embodiments, an effective dosage is provided in pulsed dosing (i.e., administration of the compound in consecutive days, followed by consecutive days of rest from administration).

In some embodiments, the disclosure provides a pharmaceutical composition for oral administration containing at least one cyclic peptide disclosed herein and a pharmaceutical excipient suitable for oral administration. The composition is in the form of a solid, liquid, gel, semi-liquid, or semi-solid. In some embodiments, the composition further comprises a second agent.

In some embodiments, this disclosure provides a solid pharmaceutical composition for oral administration containing: (i) a cyclic peptide disclosed herein; and (ii) a pharmaceutical excipient suitable for oral administration. In some embodiments, the composition further contains: (iii) a third agent or even a fourth agent. In some embodiments, each compound or agent is present in a therapeutically effective amount. In other embodiments, one or more compounds or agents is present in a sub-therapeutic amount, and the compounds or agents act synergistically to provide a therapeutically effective pharmaceutical composition.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered cyclic peptide moistened with an inert liquid diluent.

In some embodiments, the disclosure provides a pharmaceutical composition for injection containing a cyclic peptide disclosed herein and a pharmaceutical excipient suitable for injection. Components and amounts of agents in the composition are as described herein.

In certain embodiments, the forms in which the cyclic peptide disclosed herein are incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the cyclic peptide disclosed herein in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions may also be prepared from a cyclic peptide described herein and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art.

See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

The disclosure also provides kits. The kits may include a cyclic peptide disclosed herein and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the cyclic peptide disclosed herein and the agent are provided as separate compositions in separate containers within the kit. In some embodiments, the cyclic peptide disclosed herein and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Methods of Use

In one aspect, the present disclosure provides a method of inhibiting MDM2, comprising administering a cyclic peptide described herein to a subject in need thereof. In another aspect, the present disclosure provides a method of inhibiting MDM2 and MDM4, comprising administering a cyclic peptide described herein to a subject in need thereof.

In another aspect, the present disclosure provides a method of treating a disease or disorder in a subject in need thereof, comprising administering to said subject a therapeutically effective amount of a cyclic peptide described herein. In some embodiments, the method for treating the disease or disorder comprises administering to said subject an MDM2 inhibitor. In some embodiments, the method for treating the disease or disorder comprises administering to said subject an MDM2/MDM4 dual inhibitor. In some embodiments, the cyclic peptide disclosed herein is an MDM2 inhibitor. In some embodiments, the cyclic peptide disclosed herein is an MDM2/MDM4 dual inhibitor.

In some embodiments, the disease or disorder is cancer. In some embodiments, the cancer is selected from acute lymphoblastic leukemia, acute myeloid leukemia, chronic lymphocytic leukemia, and chronic myeloid leukemia. In some embodiments, the disease or disorder is associated with the proliferation of senescent cells. In some embodiments, the disease or disorder associated with the proliferation of senescent cells is selected from type 2 diabetes, Huntington's disease, non-alcoholic fatty liver disease, and hyperlipidemia. In some embodiments, the disease or disorder associated with the proliferation of senescent cells is selected from a cardiovascular disease, an inflammatory disease, an auto-immune disease, a metabolic disease, a pulmonary disease, an ophthalmic disease, an otic disease, a renal disease, and a dermatological disease.

In a further embodiment, disclosed herein is a method of treating a cancer condition, wherein the cyclic peptide disclosed herein (e.g., an MDM2 inhibitor or MDM2/MDM4 dual inhibitor) is effective in one or more method of inhibiting proliferation of cancer cells, inhibiting metastasis of cancer cells, reducing severity or incidence of symptoms associated with the presence of cancer cells, and promoting an immune response to tumor cells. In some embodiments, said method comprises administering to the cancer cells a therapeutically effective amount of a cyclic peptide disclosed herein. In some embodiments, the cancer is selected from acute lymphoblastic leukemia, acute myeloid leukemia, chronic lymphocytic leukemia, and chronic myeloid leukemia. In some embodiments, the cyclic peptide disclosed herein is an MDM2 inhibitor. In some embodiments, the cyclic peptide disclosed herein is an MDM2/MDM4 dual inhibitor. In some embodiments, the administration takes place in vitro. In some embodiments, the administration takes place in vivo.

As used herein, a therapeutically effective amount of a cyclic peptide disclosed herein refers to an amount sufficient to affect the intended application, including but not limited to, disease treatment, as defined herein. Also contemplated in the subject methods is the use of a sub-therapeutic amount of a cyclic peptide disclosed herein for treating an intended disease condition.

The amount of the cyclic peptide disclosed herein administered will vary depending upon the intended application (in vitro or in vivo), or the subject and disease condition being treated, e.g., the weight and age of the subject, the severity of the disease condition, the manner of administration and the like, which can readily be determined by one of ordinary skill in the art.

In some embodiments, therapeutic efficacy is measured based on an effect of treating a proliferative disorder, such as cancer. In general, therapeutic efficacy of the methods and compositions disclosed herein, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, the reduction in the rate of growth of a tumor, and/or a reduction in the size of at least one tumor. Several parameters to be considered in the determination of therapeutic efficacy are discussed herein. The proper combination of parameters for a particular situation can be established by the clinician. The progress of the method disclosed herein in treating cancer (e.g., reducing tumor size or eradicating cancerous cells) can be ascertained using any suitable method, such as those methods currently used in the clinic to track tumor size and cancer progress. The primary efficacy parameter used to evaluate the treatment of cancer by the method and compositions disclosed herein preferably is a reduction in the size of a tumor. Tumor size can be figured using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

In some desirable embodiments, the growth of a tumor is stabilized (i.e., one or more tumors do not increase more than 1%, 5%, 10%, 15%, or 20% in size, and/or do not metastasize) as a result of the method and compositions disclosed herein. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. Preferably, the method disclosed herein reduces the size of a tumor at least about 5% (e.g., at least about 10%, 15%, 20%, or 25%). More preferably, tumor size is reduced at least about 30% (e.g., at least about 35%, 40%, 45%, 50%, 55%, 60%, or 65%). Even more preferably, tumor size is reduced at least about 70% (e.g., at least about 75%, 80%, 85%, 90%, or 95%). Most preferably, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the efficacy of the method disclosed herein in reducing tumor size can be determined by measuring the percentage of necrotic (i.e., dead) tissue of a surgically resected tumor following completion of the therapeutic period. In some further embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is 100%, that is, no tumor tissue is present or detectable.

The efficacy of the method disclosed herein can be determined by a number of secondary parameters. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA), prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also can efficiently distinguish small tumors from surrounding tissue (see, for example, U.S. Pat. No. 4,932,412).

In additional desirable embodiments, the treatment of cancer in a human patient in accordance with the method disclosed herein is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient by the method disclosed herein. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with this disclosure can be evidenced in terms of pain intensity, analgesic consumption, and/or the Karnofsky Performance Scale score. The treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

In some embodiments, tumor size is reduced as a result of the method disclosed herein preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. Desirably, the method disclosed herein is associated with minimal adverse events, e.g. Grade 0, Grade 1, or Grade 2 adverse events, as graded by the CTEP/NCI.

However, as discussed herein, reduction of tumor size, although preferred, is not required in that the actual size of tumor may not shrink despite the eradication of tumor cells. Eradication of cancerous cells is sufficient to realize a therapeutic effect. Likewise, any reduction in tumor size is sufficient to realize a therapeutic effect.

Detection, monitoring and rating of various cancers in a human are further described in Cancer Facts and Figures 2001, American Cancer Society, New York, N.Y., and International Patent Application WO 01/24684. Accordingly, a clinician can use standard tests to determine the efficacy of the various embodiments of the method disclosed herein in treating cancer. However, in addition to tumor size and spread, the clinician also may consider quality of life and survival of the patient in evaluating efficacy of treatment.

In some embodiments, administration of a cyclic peptide disclosed herein provides improved therapeutic efficacy. Improved efficacy may be measured using any method known in the art, including but not limited to those described herein. In some embodiments, the improved therapeutic efficacy is an improvement of at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000% or more, using an appropriate measure (e.g. tumor size reduction, duration of tumor size stability, duration of time free from metastatic events, duration of disease-free survival). Improved efficacy may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold or more, using an appropriate measure (e.g. tumor size reduction, duration of tumor size stability, duration of time free from metastatic events, duration of disease-free survival).

Measuring inhibition of biological effects of MDM2 and/or MDM4 can comprise performing an assay on a biological sample, such as a sample from a subject. Any of a variety of samples may be selected, depending on the assay. Examples of samples include, but are not limited to, blood samples (e.g. blood plasma or serum), exhaled breath condensate samples, bronchoalveolar lavage fluid, sputum samples, urine samples, and tissue samples.

A subject being treated with a cyclic peptide disclosed herein may be monitored to determine the effectiveness of treatment, and the treatment regimen may be adjusted based on the subject's physiological response to treatment. For example, if inhibition of a biological effect of MDM2 and/or MDM4 inhibition is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a cyclic peptide disclosed herein is discontinued if inhibition of the biological effect is above or below a threshold, such as in a lack of response or an adverse reaction. The biological effect may be a change in any of a variety of physiological indicators.

In general, an MDM2 inhibitor is a compound that inhibits one or more biological effects of MDM2. Examples of biological effects of MDM2 include, but are not limited to, ubiquitination of p53 and inhibition of p53 transcriptional activation. Such biological effects may be inhibited by about or more than about 10%, 20%, 30%, 40%, 50%, 60%, 70%, 80%, 90%, 95%, or more.

In general, an MDM2/MDM4 dual inhibitor is a compound that inhibits one or more biological effects of MDM2 and MDM4. Examples of biological effects of MDM2 and MDM4 include, but are not limited to, ubiquitination of p53 and inhibition of p53 transcriptional activation. Such biological effects may be inhibited by about or more than about 10%, 20%, 30%, 40%, 50%, 60%, 70%, 80%, 90%, 95%, or more.

In some other embodiments, the subject methods are useful for treating a disease condition associated with MDM2. Any disease condition that results directly or indirectly from an abnormal activity or expression level of MDM2 can be an intended disease condition. In some other embodiments, the subject methods are useful for treating a disease condition associated with MDM2 and MDM4. Any disease condition that results directly or indirectly from an abnormal activity or expression level of MDM2 and MDM4 can be an intended disease condition. In some embodiments, the disease condition is a proliferative disorder, such as described herein, including but not limited to cancer. In some embodiments, the disease condition is cancer. In some embodiments, the cancer is selected from acute lymphoblastic leukemia, acute myeloid leukemia, chronic lymphocytic leukemia, and chronic myeloid leukemia.

In some embodiments, the compounds of the disclosure are administered to treat conditions other than cancer. In some embodiments, the compounds of the disclosure induce the death of a senescent cell. In some embodiments, inducing the death of a senescent cell treats a condition associated with the proliferation of senescent cells. In some embodiments, the compounds of the disclosure are administered to treat a disease or disorder associated with the proliferation of senescent cells. Exemplary disease or disorders associated with the proliferation of senescent cells include cardiovascular diseases, inflammatory or autoimmune diseases, metabolic diseases, pulmonary diseases, ophthalmic diseases, otic diseases, and dermatological diseases.

Non-limiting examples of cardiovascular diseases associated with the proliferation of senescent cells include but are not limited to atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke.

Non-limiting examples of inflammatory or autoimmune diseases associated with the proliferation of senescent cells include but are not limited to osteoarthritis, osteoporosis, inflammatory bowel disease, and herniated intervertebral discs.

Non-limited examples of metabolic diseases associated with the proliferation of senescent cells include but are not limited to diabetes, and metabolic syndrome.

Non-limiting examples of pulmonary diseases associated with the proliferation of senescent cells include but are not limited to idiopathic pulmonary fibrosis, chronic obstructive pulmonary disease, asthma, cystic fibrosis, emphysema, bronchiectasis, and loss of pulmonary function.

Non-limiting examples of ophthalmic diseases include but are not limited to cataracts, macular degeneration, glaucoma, and keratoconus.

Non-limiting examples of otic diseases associated with the proliferation of senescent cells include but are not limited to conductive hearing loss.

Non-limiting examples of dermatological diseases associated with the proliferation of senescent cells include but are not limited to eczema, psoriasis, hyperpigmentation, impaired skin wound healing, hair loss, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus.

Certain embodiments contemplate a human subject such as a subject that has been diagnosed as having or being at risk for developing or acquiring a proliferative disorder condition. Certain other embodiments contemplate a non-human subject, for example a non-human primate such as a macaque, chimpanzee, gorilla, vervet, orangutan, baboon or other non-human primate, including such non-human subjects that can be known to the art as preclinical models. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of this disclosure, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

### Combination Therapy

In some embodiments, disclosed herein are methods for further combination therapies in which, in addition to a cyclic peptide described herein, one or more second agents known to modulate other pathways, or other components of the same pathway, or even overlapping sets of target proteins is used. In one aspect, such therapy includes but is not limited to the combination of the composition comprising a cyclic peptide described herein with one or more chemotherapeutic agents, therapeutic antibodies, immunotherapeutic agents, and radiation treatment, to provide, where desired, a synergistic or additive therapeutic effect.

In some embodiments, disclosed herein are methods and pharmaceutical compositions for inhibiting abnormal cell growth in a mammal which comprises an amount of a cyclic peptide described herein, in combination with an amount of an anti-cancer agent (e.g., a chemotherapeutic agent). Many chemotherapeutics are presently known in the art and can be used in combination with the cyclic peptides disclosed herein.

In some embodiments, disclosed herein is a method for using the cyclic peptides described herein or pharmaceutical composition in combination with other tumor treatment approaches, including surgery, ionizing radiation, photodynamic therapy, or implants, e.g., with corticosteroids, hormones, or used as radiosensitizers.

### Experimental

Unless stated otherwise, all reagents were purchased from commercial suppliers without further purification. Solvent drying by standard methods was employed when necessary. The following abbreviations are used in the experimental section: COMU=(1-cyano-2-ethoxy-2-oxoethylidenaminooxy)dimethylamino-morpholino-carbenium hexafluorophosphate; DBU=1,8-diazabicyclo[5.4.0]undec-7-ene; DCM=dichloromethane; DMF=N,N-dimethylformamide; DIPEA=diisopropylethylamine; DMSO=dimethylsulfoxide; Fmoc=9-fluorenylmethoxycarbonyl; HATU=1-[bis(dimethylamino)methylene]-1H-1,2,3-triazolo[4,5-b]pyridinium 3-oxid hexafluorophosphate; HPLC=high performance liquid chromatography; MeOH=methanol; N₂=nitrogen gas; SPPS=solid phase peptide synthesis; FA=formic acid; Xaa=any amino acid; UV=ultraviolet; DIC=N,N′-diisopropylcarbodiimide; HFIP=hexafluoroisopropanol; MS=mass spectrometry; FITC=fluorescein isothiocyanate; DTT=dithiothreitol; MDM2=mouse double minute 2 homolog; HDM2=human double minute 2 homolog; FAM=fluorescein amidite; MDM4=mouse double minute 4 homolog; HDM4=human double minute 4 homolog; FBS=fetal bovine serum.

### Cyclic Peptide Synthesis

### Step 1: Loading of 2-Chlorotrityl Resin

Fmoc-Xaa (10 mmol) was dried in a vacuum desiccator over DrieRite® overnight. The dried amino acid was dissolved in dry DCM (50 mL) containing DIPEA (40 mmol) dried over molecular sieves. The reaction mixture was sonicated until Fmoc-Xaa was completely dissolved. 2-chlorotrityl resin (5 g) was added under a stream of N₂, and the reaction mixture was shaken for 4 hours. The resin was treated with a solution of 1:2:17 MeOH/DIPEA/DMF (15 mL) and shaken (3×15 minutes). The resin was washed with DMF (3×15 mL) followed by DCM (3×15 mL). The extent of resin loading was calculated by UV quantification of Fmoc release following deprotection.

### Step 2: Amino Acid Coupling

Fmoc-Xaa (4 equivalents), DIPEA (6 equivalents), and HATU (3.8 equivalents) were added to the resin in DMF (2 mL) and the reaction mixture was shaken at room temperature for 1 hour. The resin was washed with DMF (3×3 mL) followed by DCM (3×3 mL).

### Step 3: On-Resin Fmoc Deprotection

The resin was treated with a solution of 20% 4-methyl-piperidine in DMF (3 mL) and shaken at room temperature for 20 minutes. Alternatively, the resin was treated with a solution of 2% piperidine and 2% DBU in DMF (3 mL) and shaken at room temperature for 10 minutes, twice. The resin was washed with DMF (3×3 mL) followed by DCM (3×3 mL).

### Step 4: Peptoid Coupling

A 2:1 solution of 1 M bromoacetic acid/0.5 M DIC in DMF was activated with shaking for 20 minutes at room temperature. The resulting precipitate was allowed to settle and the supernatant was collected and shaken with the deprotected resin for 20 minutes at room temperature. The resin was washed with DMF (3×3 mL) followed by DCM (3×3 mL). The resin was treated with treated with a 1 M solution of amine in DMF and shaken for 1 hour at room temperature.

### Step 5: Peptide Cleavage

To cleave the completed linear peptide, the resin was treated with 5 resin volumes of 30% HFIP in DCM and shaken for 1 hour. The resin was washed with 5 resin volumes of DCM. The resin was treated with 5 resin volumes of 30% HFIP in DCM and shaken for 30 minutes.

Step 6: Cyclization with COMU

The dried linear peptide was dissolved in MeCN (2 mL) containing DIPEA (9 equivalents), and the resulting solution was added dropwise to a solution of 1:10 MeCN/DCM containing COMU (4 equivalents) to a final concentration of 1 mg crude peptide per mL. The reaction mixture was shaken at room temperature for 16 hours until complete cyclization was achieved as monitored by LCMS. The reaction mixture was concentrated under vacuum.

### Step 7: Purification of Peptides

By-products of COMU cyclization were removed via mass-directed purification on a Waters HPLC system equipped with an Xbridge BEH C18 OBD 130 Å 5 μm, 10×250 mm column eluting with H₂O/MeCN modified with 0.1% FA. Peptide purity was analyzed by HPLC-MS on a Waters HPLC system and Waters 3100 mass spectrometer equipped with a CORTECS T3 2.7 μm 4.6×50 over a gradient of H₂O/MeCN modified with 0.10% FA.

### Fluorescence Polarization Assay 1

Human MDM2 (HDM2) 1-116 (20 μL) and FITC-labeled p53 (18-26), 50 nM and 10 nM respectively in 10 mM Tris, 50 mM NaCl, 0.01% Tween20, and 1 mM DTT at pH 7.4 were dispensed into an opaque, black, 384 well plate. Compounds dissolved in DMSO were pin transferred (˜200 nL) to the 384 well plate containing the MDM2/p53 solution. After incubation for 10 minutes, the fluorescence polarization was read on a Molecular Devices SpectraMax plate reader equipped with a Fluorescein FP cartridge. In addition to probe alone (positive control) and probe/MDM2 (negative control), a titration of linear p53(18-26) was included on every plate as an additional control. IC₅₀ values were fit using either Prism or Collaborative Drug Discovery.

### Fluorescence Polarization Assay 2

Human MDM2 (HDM2) 1-116 (20 μL) and FAM-labeled RFMDYWEGL-NH₂, 50 nM and 10 nM respectively in 10 mM Tris, 50 mM NaCl, 0.010% Tween20, and 1 mM DTT at pH 7.4 were dispensed into an opaque, black, 384 well plate. Compounds dissolved in DMSO were pin transferred (˜100-200 nL) to the 384 well plate containing the MDM2/p53 solution. After incubation for 60 minutes, the fluorescence polarization was read on a Molecular Devices SpectraMax plate reader equipped with a Fluorescein FP cartridge. In addition to probe alone (positive control) and probe/MDM2 (negative control), a titration of linear p53(18-26) was included on every plate as an additional control. IC₅₀ values were fit using either Prism or Collaborative Drug Discovery.

### Fluorescence Polarization Assay 2

Human MDM4 (HDM4) 1-114 (20 μL) and FAM-labeled RFMDYWEGL-NH₂, 100 nM and 10 nM respectively in 10 mM Tris, 50 mM NaCl, 0.01% Tween20, and 1 mM DTT at pH 7.4 were dispensed into an opaque, black, 384 well plate. Compounds dissolved in DMSO were pin transferred (˜100-200 nL) to the 384 well plate containing the MDM4/p53 solution. After incubation for 60 minutes, the fluorescence polarization was read on a Molecular Devices SpectraMax plate reader equipped with a Fluorescein FP cartridge. In addition to probe alone (positive control) and probe/MDM4 (negative control), a titration of linear p53(18-26) was included on every plate as an additional control. IC₅₀ values were fit using either Prism or Collaborative Drug Discovery.

### Cellular Fluorescence Assay

MOLM-13 cells were grown in suspension in T75 flasks in RPMI medium with 10% fetal bovine serum (FBS) at 37° C. with 5% CO₂. 40 μL of MOLM-13 cells were plated into columns 1-22 of black, clear bottom, 384 well plates at a density of 1,500 cells per well in PRMI with 10% FBS. Columns 23 and 24 were filled with 40 μL of media as a positive control. 100 nL of compounds dissolved in DMSO were pin transferred into columns 3-22 of the 384 well plate. Columns 1 and 2 served as negative control wells. Plates were incubated for 72 hours at 37° C. with 5% CO₂. After incubation, the cells were given 10 μL of 2 mM Resazurin in RPMI with 10% FBS and incubated for 3 hours. The fluorescence intensity was read on a Molecular Devices SpectraMax i3x plate reader (Excitation wavelength=535 nm, Emission wavelength=585 nm).

### Parallel Artificial Membrane Permeability Assay (PAMPA)

A 96-well donor plate with 0.45 μm hydrophobic Immobilon-P membrane supports (Millipore) and a 96-well Teflon acceptor plate are used in the permeability assay. The donor wells are prepared by adding 150 μL of each cyclic peptide solution (10 μM in 5% DMSO/PBS at pH 7.4) to the wells in triplicate. A 1% (w/v) solution of lecithin in dodecane is prepared and sonicated for 5 minutes prior to use. The dodecane lecithin solution (5 μL) is applied to the membrane supports in the wells of the donor plate. The acceptor plate is prepared by adding 300 μL of 5% DMSO/PBS (pH 7.4) to each well. The donor plate is then placed on top of the acceptor plate so the artificial membrane is in contact with the buffer solution below. A lid is placed on the donor well, and the system is covered with a glass evaporating dish and left for 10 hours at room temperature. A wet paper towel is placed on the inside of the chamber to prevent evaporation.

Once the assay is complete, 100 μL from the donor and acceptor wells are aliquoted to a 96-well sample plate and sealed. Samples are analyzed on an LC/MS detector in SIM mode where the Acceptor and Donor concentrations are represented as the integration under the curve for the m/z corresponding to (exact mass+a proton) for ESI+ mode or (exact mass−a proton) for ESI-mode.

The LC/MS peak integrations are used to calculate an equilibrium value adjusted for retention (E_{R}):

E_{R}=(P_{A}V_{A}+P_{D}V_{D})/(V_{A}+V_{D})

where P_{A} is the peak integration of the acceptor, V_{A} is the volume of the acceptor (cm³), P_{D} is the analyte-to-standard peak of the donor, and V_{D} is the volume of the donor.

Transmittance percentage (% T) is calculated for each sample:

% T=(P_{A}/E_{R})×100

and the % T values are converted into time-independent Pe values:

Pe=[(V_{A}×V_{D})/(V₀×A×t)]×1n(1−(% T/100))

where V₀ is the total volume (cm³), A is the accessible filter area of the membrane (0.24 cm²), and t is the incubation time (s). Average % T and Pe values are calculated for each compound from at least three data points excluding extreme outlying permeability values. Standard deviations are calculated for the average values. Because percent recovery is accounted for in the E_{R}, it is assumed for the calculation that there is no compound loss.

### MOLM-13 Mouse Xenograft Model

110 female nu/nu mice were subcutaneously injected in the lower left abdominal flank with MOLM-13 cells (5×10⁻⁶ cells per animal in 200 μL 1:1 PBS/Matrigel). Mice were divided into 8 treatment groups and administered either vehicle (5% ethanol, 12.5% Solutol HS, 12.5% PEG300, and 70% 50 mM PBS), idasanutlin, or Compound 35 according to Table 1:

TABLE 1

| | | | Dose | | Treatments |
|---|---|---|---|---|---|
| Group | Treatment | Route | (mg/kg) | N | Per Week |
| 1 | Vehicle | IV | 0 | 10 | 3 |
| 2 | Vehicle | IP | 0 | 10 | 7 |
| 3 | Vehicle | PO | 0 | 10 | 7 |
| 4 | Idasanutlin | PO | 30 | 10 | 7 |
| 5 | Cmpd. 35 | IV | 20 | 10 | 3 |
| 6 | Cmpd. 35 | IV | 50 | 10 | 3 |
| 7 | Cmpd. 35 | IP | 20 | 10 | 7 |
| 8 | Cmpd. 35 | IP | 50 | 10 | 7 |

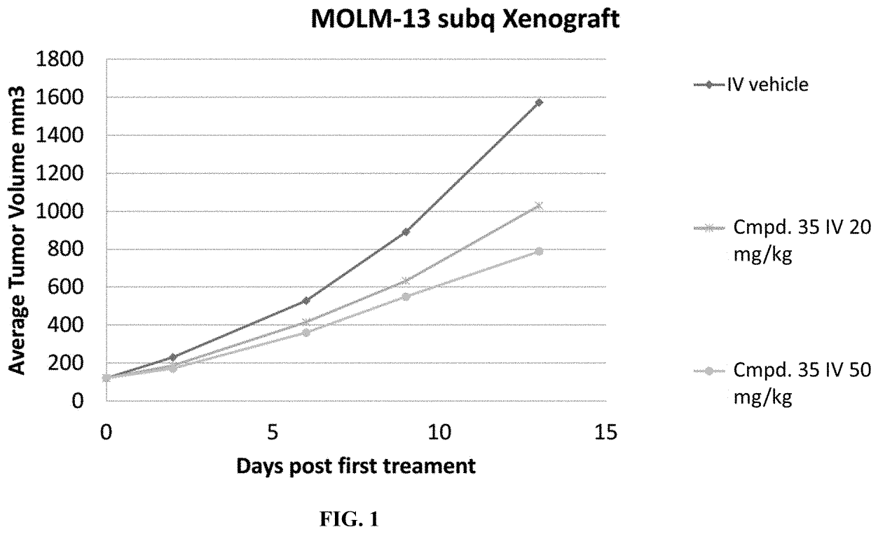

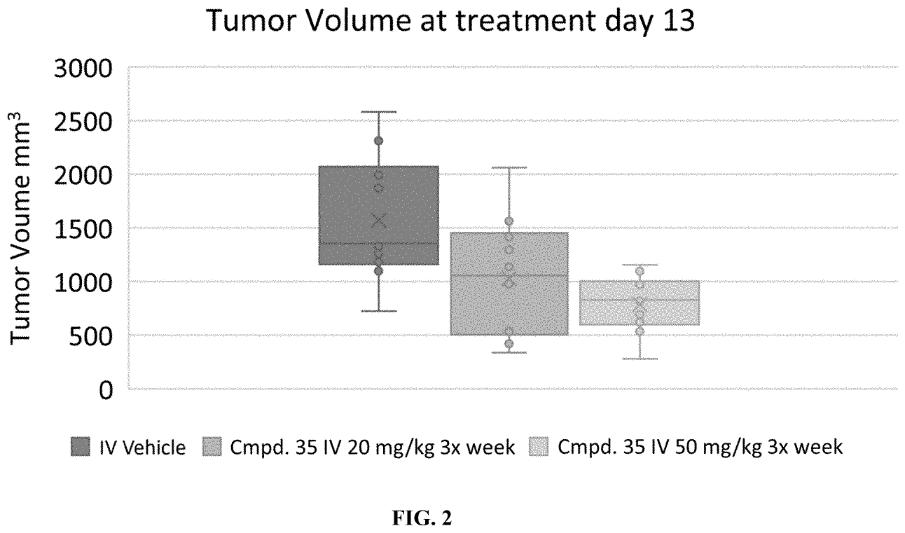

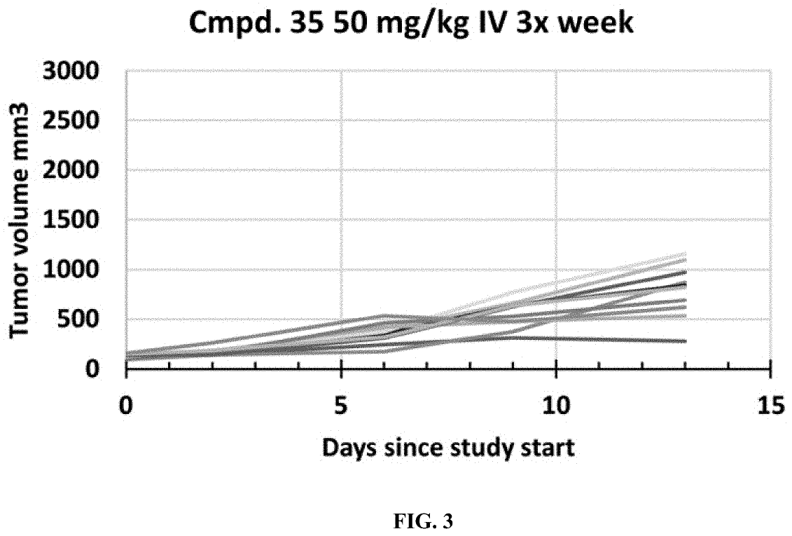

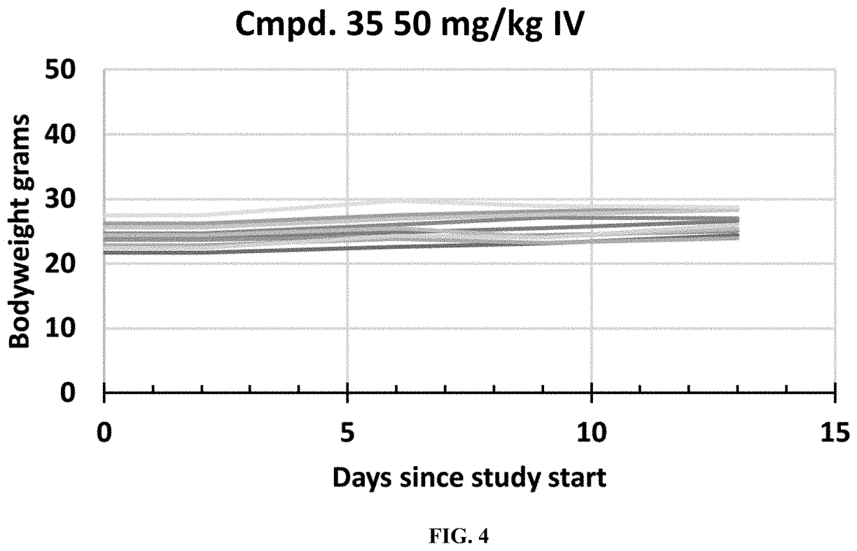

The mice were monitored for 2 weeks. The change in average tumor volume over time for intravenous Compound 35 versus vehicle is shown in FIG. 1. The tumor volume at treatment day 13 for intravenous Compound 35 versus vehicle is shown in FIG. 2. The change in tumor volume over time for each mouse treated with intravenous Compound 35 is shown in FIG. 3. The change in body weight over time for each mouse treated with intravenous Compound 35 is shown in FIG. 4.

### Pharmacokinetic Parameters of Compound 35

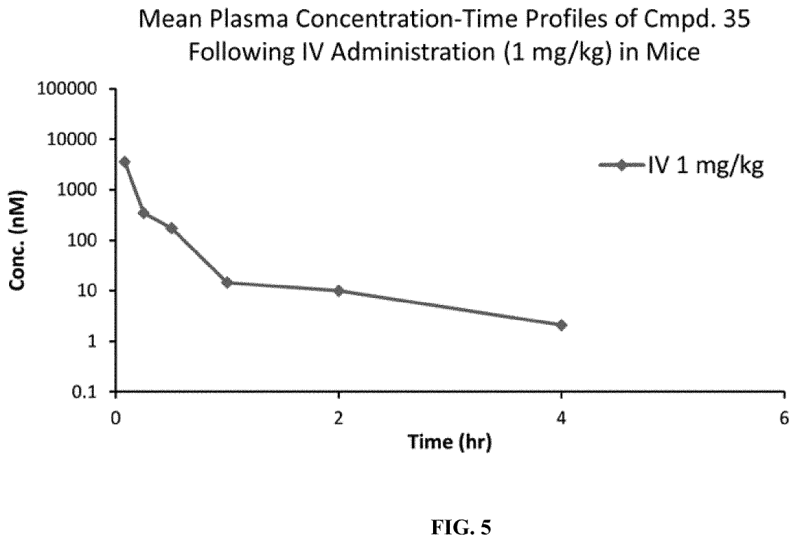

Mice were treated intravenously with Compound 35 (1 mg/kg) and plasma was collected at various timepoints to determine pharmacokinetic parameters. The change in mean plasma concentration over time is shown in FIG. 5, and all pharmacokinetic parameters obtained are summarized in Table 2:

TABLE 2

| Tₘₐₓ | Cₘₐₓ | T_{1/2} | MRTₗₐₛₜ | MRT_{inf} | AUCₗₐₛₜ | AUC_{inf} | Cl | Vₛₛ |
|---|---|---|---|---|---|---|---|---|
| (hr) | (ng/mL) | (hr) | (hr) | (hr) | (hr * ng/mL) | (hr * ng/mL) | (mL/hr/kg) | (mL/kg) |
| 0.08 | 3570 | 1.04 | 0.13 | 0.15 | 1040 | 1040 | 959 | 140 |

In some embodiments, the cyclic peptide described herein is a cyclic peptide depicted in Table 3:

TABLE 3

| | | MDM2 |
|---|---|---|
| | | IC₅₀ |
| No. | SMILES String** | (μM)* |
| 1 | [H]OC(=O)CC[C@@H]1NC(=O)[C@H](Cc2cn | A |
| | ([H])c3ccccc23)NC(=O)[C@H](CC(=O)O[H])NC | |
| | (=O)[C@H](CC(C)C)NC(=O)[C@H](Cc2c[nH]c3cc | |
| | (Cl)ccc23)NC(=O)[C@H](CCC(=O)O[H])NC(=O) | |
| | [C@H](Cc2ccccc2)NC(=O)[C@@H]2CCCN2C(=O) | |
| | [C@H]2CCCN2C(=O)[C@H](Cc2ccccc2)NC1=O | |
| 2 | [H]OC(=O)CC[C@@H]1NC(=O)[C@H](Cc2cn | B |
| | ([H])c3ccccc23)NC(=O)[C@H](CC(=O)O[H])NC | |
| | (=O)[C@H](CC(C)C)NC(=O)[C@H](Cc2cn([H]) | |
| | c3ccccc23)NC(=O)[C@H](CCC(=O)O[H])NC(=O) | |
| | [C@H](Cc2ccccc2)NC(=O)[C@@H]2CCCN2C(=O) | |
| | [C@H]2CCCN2C(=O)[C@H](Cc2ccccc2)NC1=O | |
| 3 | [H]OC(=O)CC[C@@H]1NC(=O)[C@H](Cc2ccccc2) | A |
| | NC(=O)[C@@H]2CCCN2C(=O)[C@H] | |
| | 2CCCN2C(=O)[C@H](Cc2ccccc2)NC | |
| | (=O)[C@H](CCC(=O)O[H])NC | |
| | (=O)[C@H](Cc2cn([H])c3ccccc23)NC(=O)[C@H](C | |
| | C(=O)O[H])NC(=O)[C@H](CC(C)C)N(C)C(=O) | |
| | [C@H](Cc2cn([H])c3ccccc23)NC1=O | |
| 4 | [H]OC(=O)CC[C@@H]1NC(=O)[C@H](Cc2ccccc2) | A |
| | NC(=O)[C@@H]2CCCN2C(=O)[C@H]2CCCN2C | |
| | (=O)[C@H](Cc2ccc(Cl)cc2)NC(=O)[C@H](CCC | |
| | (=O)O[H])NC(=O)[C@H](Cc2ccc(F)cc2)NC(=O) | |
| | [C@H](CC(=O)O[H])NC(=O)[C@H](CC(C)C) | |
| | N(C)C(=O)[C@H](Cc2cn([H])c3ccccc23)NC1=O | |
| 5 | [H]OC(=O)CC[C@@H]1NC(=O)[C@H](Cc2cccs2) | A |
| | NC(=O)[C@@H]2CCCN2C(=O)[C@H] | |
| | 2CCCN2C(=O)[C@H](Cc2ccccc2)NC(=O) | |
| | [C@H](CCC(=O)O[H])NC(=O) | |
| | [C@H](Cc2ccc(F)cc2)NC(=O)[C@H](CC(=O)O[ | |
| | H])NC(=O)[C@H](CC(C)C)N(C)C(=O)[C@H] | |
| | (Cc2cn([H])c3ccccc23)NC1=O | |
| 6 | [H]OC(=O)CC[C@@H]1NC(=O)[C@H](Cc2cn([H]) | A |
| | c3ccccc23)NC(=O)[C@H](CC(=O)O[H])NC(=O) | |
| | [C@H](CC(C)C)N(C)C(=O)[C@H](Cc2ccc(Cl)cc2) | |
| | NC(=O)[C@H](CCC(=O)O[H])NC(=O)[C@H] | |
| | (Cc2ccc(F)cc2)NC(=O)[C@@H]2CCCN2C | |
| | (=O)[C@H]2CCCN2C(=O)[C@H](Cc2cccc | |
| | c2)NC1=O | |
| 7 | [H]OC(=O)C[C@@H]1NC(=O)[C@H](CC(C)C) | D |
| | N(C)C(=O)[C@H](Cc2cn([H])c3ccccc23) | |
| | NC(=O)[C@H](CCCC)N(C)C(=O)[C@H] | |
| | (Cc2ccccc2)NC(=O)[C@@H]2CCCN2C | |
| | (=O)[C@H]2CCCN2C(=O)[C@H](Cc2cccc | |
| | c2)NC(=O)[C@H](CCCC)N(C)C(=O)[C@H] | |
| | (Cc2cn([H])c3ccccc23)NC1=O | |
| 8 | [H]OC(=O)CC[C@@H]1NC(=O)[C@H](Cc2ccccc2) | C |
| | NC(=O)[C@@H]2CCCN2C(=O)[C@H]2CCCCN2C | |
| | (=O)[C@H](Cc2ccc(Cl)cc2)NC(=O)[C@H] | |
| | (C)N(C)C(=O)[C@H](Cc2ccc(F)cc2)NC(=O) | |
| | [C@H](C)N(C)C(=O)[C@H]2CCCN2C | |
| | (=O)[C@H](Cc2ccc(O[H])cc2)NC1=O | |
| 9 | [H]OC(=O)CC[C@@H]1NC(=O)[C@H](Cc2ccccc2) | A |
| | NC(=O)[C@@H]2CCCN2C(=O)[C@H]2CCCCN2C | |
| | (=O)[C@H](Cc2ccccc2)NC(=O)[C@H] | |
| | (CCC(=O)O[H])NC(=O)[C@H](Cc2ccc(F)cc2)NC | |
| | (=O)[C@H](C)N(C)C(=O)[C@H]2CCCN2C(=O) | |
| | [C@H](Cc2cn([H])c3ccccc23)NC1=O | |
| 10 | [H]Oc1ccc(C[C@@H]2NC(=O)[C@@H](NC(=O) | B |
| | [C@H](Cc3ccccc3)NC(=O)[C@@H]3CCCN3C(=O) | |
| | [C@H]3CCCCN3C(=O)[C@H](Cc3ccc | |
| | (Cl)cc3)NC(=O)[C@H](C)N(C)C | |
| | (=O)[C@H](Cc3ccc(F)cc3)NC(=O)[C@ | |
| | H](C)N(C)C(=O)[C@H]3CCCN3C2=O)C(C)(C)C)cc1 | |
| 11 | [H]Oc1ccc(C[C@@H]2NC(=O)[C@@H](NC(=O) | D |
| | [C@H](Cc3ccccc3)NC(=O)[C@@H]3CCCN3C | |
| | (=O)[C@H]3CCCCN3C(=O)[C@H] | |
| | (Cc3ccc(Cl)cc3)NC(=O)[C@H] | |
| | (C)N(C)C(=O)[C@H](Cc3ccc(F)cc3)NC(=O)[C@ | |
| | H](C)N(C)C(=O)[C@H]3CCCN3C2=O)C(C)(C)C)cc1 | |
| 12 | [H]Oc1ccc(C[C@@H]2NC(=O)[C@@H](NC(=O) | C |
| | [C@H](Cc3ccccc3)NC(=O)[C@@H]3CCCN3C(=O) | |
| | [C@H]3CCCCN3C(=O)[C@H] | |
| | (Cc3ccc(Cl)cc3)NC(=O) | |
| | [C@H](C)N(C)C(=O)[C@H](Cc3ccc(F)cc3)NC(=O) | |
| | [C@H](C)N(C)C(=O)CN(CCOC)C2=O)C(C)(C)C)cc1 | |
| 13 | [H]Oc1ccc(C[C@@H]2NC(=O)[C@@H](NC(=O) | C |
| | [C@H](Cc3ccccc3)NC(=O)[C@@H]3CCCN3C(=O) | |
| | [C@H]3CCCCN3C(=O)[C@H] | |
| | (Cc3ccc(Cl)cc3)NC(=O) | |
| | [C@H](C)N(C)C(=O)[C@H](Cc3ccc(F)cc3)NC(=O) | |
| | [C@H](C)N(C)C(=O)CN(CCOC)C2=O)C(C)(C)C)cc1 | |
| 14 | [H]Oc1ccc(C[C@@H]2NC(=O)[C@@H](NC(=O) | B |
| | [C@H](Cc3ccccc3)NC(=O)[C@@H]3CCCN3C(=O) | |
| | [C@@H](C(C)C)N(C)C(=O) | |
| | [C@H](Cc3ccc(Cl)cc3)NC(=O) | |
| | [C@H](C)N(C)C(=O)[C@H](Cc3ccc(F)cc3)NC(=O) | |
| | [C@H](C)N(C)C(=O)CN(CCOC)C2=O)C(C)(C)C)cc1 | |
| 15 | [H]OC(=O)CC[C@@H]1NC(=O)[C@H](Cc2ccc | B |
| | (O[H])cc2)NC(=O)[C@@H]2CCCN2C(=O)[C@H] | |
| | 2CCCCN2C(=O)[C@H](Cc2ccc(Cl)cc2)NC(= | |
| | O)[C@H](C)N(C)C(=O)[C@H](Cc2ccc(F)cc2)NC | |
| | (=O)[C@H](C)N(C)C(=O)[C@H]2CCCN2C(=O) | |
| | [C@H](Cc2cccnc2)NC1=O | |
| 16 | [H]OC(=O)CC[C@@H]1NC(=O)[C@H](Cc2cccnc2) | A |
| | NC(=O)[C@@H]2CCCN2C(=O)[C@H]2CCCCN2C | |
| | (=O)[C@H](Cc2ccc(Cl)cc2) | |
| | NC(=O)[C@H](C)N(C)C(=O)[C@H](Cc2ccc | |
| | (F)cc2)NC(=O)[C@H](C)N(C)C(=O)[C@H] | |
| | 2CCCN2C(=O)[C@H](Cc2ccc(O[H])cc2)NC1=O | |
| 17 | COCCN1CC(=O)N(C)[C@@H](C)C(=O)N[C@@H] | A |
| | (Cc2ccc(F)cc2)C(=O)N(C)[C@@H](C)C(=O)N | |
| | [C@@H](Cc2ccc(Cl)cc2)C(=O)N(C)[C@H] | |
| | (C(C)C)C(=O)N2CCC[C@H]2C(=O)N[C@@H] | |
| | (Cc2cccs2)C(=O)N[C@H](C(=O)N | |
| | [C@@H](Cc2cccnc2)C1=O)C(C)(C)C | |
| 18 | COCCN1CC(=O)N[C@@H](Cc2ccc(Cl)cc2)C | A |
| | (=O)N2CCCC[C@@H]2C(=O)N2CCC[C@H]2C | |
| | (=O)N[C@@H](Cc2ccccc2)C(=O)N[C@H](C(= | |
| | O)N[C@@H](Cc2cccnc2)C(=O)N2CCC[C@@H] | |
| | 2C(=O)N(C)[C@@H](C)C(=O)N[C@@H] | |
| | (Cc2ccc(F)cc2)C1=O)C(C)(C)C | |
| 19 | C[C@@H]1N(C)C(=O)[C@H]2CCCN2C(=O) | A |
| | [C@H](Cc2cccnc2)NC(=O)C2CN(C2)C(=O)[C@H] | |
| | (Cc2ccccc2)NC(=O)[C@@H]2CCCN2C(=O) | |
| | [C@H]2CCCCN2C(=O)[C@H](Cc2ccc(Cl)cc2) | |
| | NC(=O)[C@@H](NC(=O)[C@H](Cc2ccc(F)cc2) | |
| | NC1=O)C(C)(C)C | |
| 20 | COCCN1CC(=O)N(C)[C@@H](C)C(=O)N[C@@H] | A |
| | (Cc2ccc(F)cc2)C(=O)N(C)[C@@H](C)C(=O)N | |
| | [C@@H](Cc2ccc(Cl)cc2)C(=O)N2CCCC[C | |
| | @@H]2C(=O)N2CCC[C@H]2C(=O)N[C@@H] | |
| | (Cc2cccs2)C(=O)N[C@H](C(=O)N[C@@H] | |
| | (Cc2cccnc2)C1=O)C(C)(C)C | |
| 21 | COCCN1CC(=O)N(C)[C@@H](C)C(=O)N[C@@H] | B |
| | (Cc2ccc(F)cc2)C(=O)N(C)[C@@H](C)C(=O)N | |
| | [C@@H](Cc2ccc(Cl)cc2)C(=O)N2CCCC[C | |
| | @@H]2C(=O)N2CCC[C@H]2C(=O)N[C@@H] | |
| | (Cc2ccccc2)C(=O)N[C@H](C(=O)N[C@@H] | |
| | (Cc2cccnc2)C1=O)C(C)(C)C | |
| 22 | COCCN1CC(=O)N(C)[C@@H](C)C(=O)N[C@@H] | B |
| | (Cc2ccc(F)cc2)C(=O)N(C)[C@@H](C)C(=O)N | |
| | [C@@H](Cc2ccc(Cl)cc2)C(=O)N2CCCC[C | |
| | @@H]2C(=O)N2CCC[C@H]2C(=O)N[C@@H] | |
| | (Cc2ccccc2)C(=O)N[C@H](C(=O)N[C@@H] | |
| | (Cc2cccc(F)c2)C1=O)C(C)(C)C | |
| 23 | COCCN1CC(=O)N(C)[C@@H](C)C(=O)N[C@@H] | A |
| | (Cc2ccc(F)cc2)C(=O)N(C)[C@@H](C)C(=O)N | |
| | [C@@H](Cc2ccc(Cl)cc2)C(=O)N(C)[C@H] | |
| | (C(C)C)C(=O)N2CCC[C@H]2C(=O)N[C@@H] | |
| | (Cc2ccccc2)C(=O)N[C@H](C(=O)N[C@@H] | |
| | (Cc2cccnc2)C1=O)C(C)(C)C | |
| 24 | COCCN1CC(=O)N(C)[C@@H](C)C(=O)N[C@@H] | B |
| | (Cc2ccc(F)cc2)C(=O)N(C)[C@@H](C)C(=O)N | |
| | [C@@H](Cc2ccc(Cl)cc2)C(=O)N(C)[C@H] | |
| | (C(C)C)C(=O)N2CCC[C@H]2C(=O)N[C@@H] | |
| | (Cc2ccccc2)C(=O)N[C@H](C(=O)N[C@@H] | |
| | (Cc2cccc(F)c2)C1=O)C(C)(C)C | |
| 25 | COCCN1CC(=O)N[C@@H](Cc2ccc(Cl)cc2)C(=O) | A |
| | N2CCCC[C@@H]2C(=O)N2CCC[C@H]2C(=O)N | |
| | [C@@H](Cc2cccs2)C(=O)N[C@H](C(=O)N[C@@H] | |
| | (Cc2cccnc2)C(=O)N2CCC | |
| | [C@@H]2C(=O)N(C)[C@@H] | |
| | (C)C(=O)N[C@@H](Cc2ccc(F)cc2)C1=O)C(C)(C)C | |
| 26 | C[C@@H]1N(C)C(=O)[C@H]2CCCN2C(=O)[C@H] | A |
| | (Cc2cccnc2)NC(=O)[C@H](C)N(C)C(=O)[C@H] | |
| | (Cc2ccccc2)NC(=O)[C@@H]2CCCN2C(=O)[C@H] | |
| | 2CCCCN2C(=O)[C@H](Cc2ccc(Cl)cc2) | |
| | NC(=O)[C@@H](N | |
| | C(=O)[C@H](Cc2ccc(F)cc2)NC1=O)C(C)(C)C | |
| 27 | CC(C)[C@@H]1N(C)C(=O)[C@H](Cc2ccccc2) | B |
| | NC(=O)[C@@H]2CCCN2C(=O)[C@H] | |
| | 2CCCCN2C(=O)[C@H](Cc2ccc(Cl)cc2)NC(=O) | |
| | [C@@H](NC(=O)[C@H](Cc2ccc(F)cc2) | |
| | NC(=O)[C@H](C)N(C)C(=O)[C@H]2CCCN2C | |
| | (=O)[C@H](Cc2cccnc2)NC1=O)C(C)(C)C | |
| 28 | C[C@@H]1N(C)C(=O)[C@H]2CCCCN2C(=O) | B |
| | [C@H](Cc2cccnc2)NC(=O)[C@@H](NC | |
| | (=O)[C@H](Cc2ccccc2)NC(=O)[C@@H] | |
| | 2CCCN2C(=O)[C@H]2CCCCN2C | |
| | (=O)[C@H](Cc2ccc(Cl)cc2)NC(=O)[C@H](C)N( | |
| | C)C(=O)[C@H](Cc2ccc(F)cc2)NC1=O)C(C)(C)C | |
| 29 | COCCN1CC(=O)N(C)[C@@H](C)C(=O)N[C@@H] | A |
| | (Cc2ccc(F)cc2)C(=O)N[C@H](C(=O)N[C@@H] | |
| | (Cc2ccc(Cl)cc2)C(=O)N2CCCC[C@@H]2 | |
| | C(=O)N2CCC[C@H]2C(=O)N[C@@H](Cc2ccccc2) | |
| | C(=O)N[C@H](C(=O)N[C@@H](Cc2cccnc2) | |
| | C1=O)C(C)(C)C)C(C)(C)C | |
| 30 | [H]Oc1ccc(C[C@@H]2NC(=O)[C@@H](NC(=O) | C |
| | [C@H](Cc3ccccc3)NC(=O)[C@@H]3CCCN3C | |
| | (=O)[C@H]3CCCCN3C(=O)[C@H](Cc3ccc( | |
| | Cl)cc3)NC(=O)[C@H](C)N(C)C(=O)[C@H] | |
| | (Cc3ccc(F)cc3)NC(=O)[C@ | |
| | H](C)N(C)C(=O)CN(CCOC)C2=O)C(C)(C)C)cc1 | |
| 31 | [H]Oc1ccc(C[C@@H]2NC(=O)[C@@H](NC(=O) | D |
| | [C@H](Cc3ccccc3)NC(=O)[C@@H]3CCCN3C | |
| | (=O)[C@@H](C(C)C)N(C)C(=O)[C@H](Cc3 | |
| | ccc(Cl)cc3)NC(=O)[C@H](C)N(C)C(=O) | |
| | [C@H](Cc3ccc(F)cc3)NC(=O) | |
| | [C@H](C)N(C)C(=O)CN(CCOC)C2=O)C(C)(C)C)cc1 | |

*A < 1.0 μM; 1.0 μM ≤ B < 2.5 μM; 2.5 μM ≤ C < 5.0 μM; 5.0 μM ≤ D
**SMILES string generated from chemical structure in ChemDraw version 19.1.

In some embodiments, the cyclic peptide described herein is a cyclic peptide depicted in Table 4.

TABLE 4

| | | MDM2 | MDM4 | MOLM- |
|---|---|---|---|---|
| | | IC₅₀ | IC₅₀ | 13 IC₅₀ |
| No. | SMILES String*** | (nM)* | (nM)** | (nM)** |
| 32 | [H]Oc1ccc(C[C@@H]2NC | A | D | B |
| | (=O)[C@H](C)N(C)C | | | |
| | (=O)[C@H](CCOC)NC(=O) | | | |
| | [C@H]3CCCN3C(=O) | | | |
| | [C@H](Cc3ccccc3)N(C)C | | | |
| | (=O)[C@H](Cc3ccc(Cl)cc3) | | | |
| | NC(=O)[C@@H](NC(=O) | | | |
| | [C@H](Cc3ccc(F)cc3)NC | | | |
| | (=O)CN(CCOC)C(=O)[ | | | |
| | C@@H](C)N(C)C2=O) | | | |
| | C(C)(C)C)cc1 | | | |
| 33 | COCC[C@@H]1NC(=O) | A | B | D |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | CN(C)C(=O)[C@H](CC2=C | | | |
| | C=C(F)C=C2)NC(=O) | | | |
| | [C@H](C)N(C)C(=O) | | | |
| | [C@@H](C)N(C)C(=O)[C@H] | | | |
| | (CC2=CSC=N2)NC | | | |
| | (=O)[C@H](C)N(C) | | | |
| | C1=O | | | |
| 34 | COCC[C@@H]1NC(=O) | B | D | B |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@H](C)N(C)C(=O)[C@H] | | | |
| | (CC2=CC=C(F)C=C2) | | | |
| | NC(=O)[C@H](C) | | | |
| | N(C)C(=O)[C@@H] | | | |
| | (C)N(C)C(=O) | | | |
| | C@H](CC2=CSC=N2) | | | |
| | NC(=O)[C@H] | | | |
| | (C)N(C)C1=O | | | |
| 35 | COCC[C@@H]1NC(=O) | C | D | C |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@@H](NC(=O)[C@H](CC | | | |
| | 2=CC=C(F)C=C2)NC(=O) | | | |
| | CN(CCOC)C(=O) | | | |
| | [C@@H](C)N(C)C(=O) | | | |
| | [C@H](CC2=CSC=N2) | | | |
| | NC(=O)[C@H](C)N(C) | | | |
| | C1=O)C(C)(C)C | | | |
| 36 | COCC[C@@H]1NC(=O) | A | B | B |
| | [C@H]2CCCN2C(=O) | | | |
| | [C@H](Cc2ccccc2)N(C)C(= | | | |
| | O)[C@H](Cc2ccc(Cl)cc2) | | | |
| | NC(=O)[C@@H](NC | | | |
| | (=O)[C@H](Cc2ccc(F)cc2)N | | | |
| | C(=O)[C@H](C)N(C)C(=O) | | | |
| | [C@@H](C)N(C)C(=O) | | | |
| | [C@H](CC2=CSC=N2) | | | |
| | NC(=O)[C@H](C)N(C) | | | |
| | C1=O)C(C)(C)C | | | |
| 37 | COCC[C@@H]1NC(=O) | B | B | B |
| | [C@H]2CCCN2C(=O) | | | |
| | [C@H](Cc2ccc(F)cc2)N(C)C | | | |
| | (=O)[C@H](CC2=CC=C | | | |
| | (Cl)C=C2)NC(=O)[C@@H] | | | |
| | (NC(=O)[C@H](Cc2ccc | | | |
| | (F)cc2)NC(=O)CN(CCOC) | | | |
| | C(=O)[C@@H](C)N | | | |
| | (C)C(=O)[C@H](CC2=CSC= | | | |
| | N2)NC(=O)[C@H](C)N(C) | | | |
| | C1=O)C(C)(C)C | | | |
| 38 | COCC[C@@H]1NC(=O) | B | B | B |
| | [C@H]2CCCN2C(=O) | | | |
| | [C@H](Cc2ccc(F)cc2)N(C)C | | | |
| | (=O)[C@H](CC2=CC=C | | | |
| | (Cl)C=C2)NC(=O) | | | |
| | [C@@H](NC(=O)[C@H] | | | |
| | (Cc2ccc(F)cc2)NC | | | |
| | (=O)[C@H](C)N(C)C(=O)[ | | | |
| | C@@H](C)N(C)C(=O) | | | |
| | [C@H](CC2=CSC=N2)NC | | | |
| | (=O)[C@H](C)N(C)C1=O) | | | |
| | C(C)(C)C | | | |
| 39 | COCC[C@@H]1NC(=O) | A | A | C |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@@H](NC(=O)[C@H](CC | | | |
| | 2=CC=C(F)C=C2)NC(=O) | | | |
| | CN(CCOC)C(=O) | | | |
| | [C@@H](C)N(C)C(=O) | | | |
| | [C@H](CC2=CSC=N2) | | | |
| | NC(=O)[C@H](C)N(C) | | | |
| | C1=O)C(C)O | | | |
| | COCC[C@@H]1NC | A | A | C |
| | ([C@H]2CCCN2C([C@@H] | | | |
| | (N(C([C@@H](NC([C@H] | | | |
| | ([C@H](O)C)NC([C@@H] | | | |
| | (NC(CN(C([C@H] | | | |
| | (N(C([C@@H](NC([C@@H] | | | |
| | (N(C1=O)C)C)=O)CC3= | | | |
| | CSC=N3)=O)C)C)=O) | | | |
| | CCOC)=O)CC4=CC=C(C=C | | | |
| | 4)F)=O)=O)CC5=CC=C | | | |
| | (C=C5)Cl)=O) | | | |
| | C)CC6=CC=CC=C6)=O)=O | | | |
| 40 | COCC[C@@H]1NC(=O) | B | D | C |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@H](C)N(C)C(=O)[C@H]( | | | |
| | CC2=CC=C(F)C=C2)NC | | | |
| | (=O)CN(CCOC)C(=O) | | | |
| | [C@@H](C)N(C)C(=O)[C | | | |
| | @H](CC2=CSC=N2)NC(=O) | | | |
| | [C@H](C)N(C)C1=O | | | |
| 41 | COCC[C@@H]1NC(=O) | A | A | C |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@@H](NC(=O)[C@H](CC | | | |
| | 2=CC=C(F)C=C2)NC(=O) | | | |
| | CN(CCOC)C(=O) | | | |
| | [C@@H](C)N(C)C(=O)[C@H] | | | |
| | (CC2=CSC=N2)NC(=O) | | | |
| | [C@H](C)N(C) | | | |
| | C1=O)C(C)C | | | |
| 42 | CC[C@@H]1NC(=O) | B | C | D |
| | [C@H](CC2=CC=C(F) | | | |
| | C=C2)NC(=O)CN(CCOC)C(= | | | |
| | O)[C@@H](C)N(C)C(=O) | | | |
| | [C@H](CC2=CSC=N2) | | | |
| | NC(=O)[C@H](C)N(C)C | | | |
| | (=O)[C@H](CCOC)NC | | | |
| | (=O)[C@H]2CCCN2C | | | |
| | (=O)[C@H](CC2=CC= | | | |
| | CC=C2)N(C)C(=O) | | | |
| | [C@H](CC2=CC=C | | | |
| | (Cl)C=C2)NC1=O | | | |
| 43 | COCC[C@@H]1NC(=O) | B | C | D |
| | [C@H]2CCCN2C(=O) | | | |
| | [C@H](CC2=CC=CC=C2)N( | | | |
| | C)C(=O)[C@H](CC2=CC= | | | |
| | C(Cl)C=C2)NC(=O) | | | |
| | [C@H](C(C)C)N(C)C(=O)[C | | | |
| | @H](CC2=CC=C(F)C=C2) | | | |
| | NC(=O)CN(CCOC)C | | | |
| | (=O)[C@@H](C)N(C)C(=O) | | | |
| | [C@H](CC2=CSC=N2)NC | | | |
| | (=O)[C@H](C)N(C)C1=O | | | |
| 44 | CCN1[C@@H](Cc2ccc | C | D | C |
| | (F)cc2)C(=O)N2CCC | | | |
| | [C@@H]2C(=O)N[C@@H](C | | | |
| | COC)C(=O)N(C)[C@@H] | | | |
| | (C)C(=O)N[C@@H] | | | |
| | (Cc2cscn2)C(=O)N(C)[C@H] | | | |
| | (C)C(=O)N(CCOC)CC(=O) | | | |
| | N[C@@H](Cc2ccc(F)cc2) | | | |
| | C(=O)N[C@H](C(=O) | | | |
| | N[C@@H](Cc2ccc(Cl)cc2) | | | |
| | C1=O)C(C)(C)C | | | |
| 45 | COCC[C@@H]1NC(=O) | A | B | B |
| | [C@H]2CCCN2C(=O) | | | |
| | [C@H](Cc2ccc(F)cc2)N(C)C | | | |
| | (=O)[C@H](Cc2ccc(Cl)cc2) | | | |
| | NC(=O)[C | | | |
| | @H]([C@@@H](C)O) | | | |
| | N(C)C(=O)[C@H] | | | |
| | (Cc2ccc(F)cc2)NC(=O) | | | |
| | CN(CCOC)C(=O)[C@@H] | | | |
| | (C)N(C)C(=O)[C@H](Cc | | | |
| | 2cscn2)NC(=O)[C@H](C) | | | |
| | N(C)C1=O | | | |
| 46 | COCC[C@@H]1NC(=O) | C | C | C |
| | [C@H]2CCCN2C(=O)[C@H] | | | |
| | (Cc2ccc(F)cc2)N(C)C | | | |
| | (=O)[C@H](Cc2ccc(Cl) | | | |
| | cc2)NC(=O)[C@@H] | | | |
| | 2CCCCN2C(=O)[C@H](Cc2cc | | | |
| | c(F)cc2)NC(=O)CN(CCOC) | | | |
| | C(=O)[C@@H](C)N(C)C | | | |
| | (=O)[C@H](Cc2cscn | | | |
| | 2)NC(=O)[C@H](C)N(C)C1=O | | | |
| 47 | COCC[C@@H]1NC | B | C | C |
| | ([C@H]2CCCN2C([C@@H] | | | |
| | (N(C([C@@H](NC([C@ | | | |
| | @H]3CCCN3C([C@@H] | | | |
| | (NC(CN(C([C@H](N(C | | | |
| | ([C@@H](NC([C@@H](N | | | |
| | (C1=O)C)C)=O)Cc4ncsc4)= | | | |
| | O)C)C)=O)CCOC)=O) | | | |
| | Cc5ccc(F)cc5)=O)=O)Cc6 | | | |
| | ccc(Cl)cc6)=O)C)Cc7ccc | | | |
| | (F)cc7)=O)=O | | | |

*A < 25.0 nM; 25.0 nM ≤ B < 50.0 nM; 50.0 nM ≤ C < 100.0 nM; 100.0 nM ≤ D
**A < 50.0 nM; 50.0 nM ≤ B < 100.0 nM; 100.0 nM ≤ C < 150.0 nM; 150.0 nM ≤ D
***SMILES string generated from chemical structure in ChemDraw version 19.1.

1. A cyclic peptide comprising:
nine to eleven amino acid residues independently selected from amino acid residues that are not charged at physiological pH;
a first and a second beta hairpin region;
and characterized by one of the following:
at least four amino acid residues comprising rings independently selected from optionally substituted monocyclic carbocycle and optionally substituted monocyclic heterocycle, wherein at least one of the monocyclic carbocycle and monocyclic heterocycle are substituted;
at least four amino acid residues with side chains selected from -alkylene-(monocyclic carbocycle) and -alkylene-(monocyclic heterocycle), wherein the monocyclic carbocycle and monocyclic heterocycle are independently optionally substituted; and
at least three amino acid residues comprising rings independently selected from optionally substituted phenyl and optionally substituted monocyclic heteroaryl.

2. (canceled)

3. The cyclic peptide of claim 1, wherein the first beta hairpin region comprises two contiguous residues independently selected from: L-Pro, D-Pro, L-Aze, D-Pip, L-NMe-Phe, and D-NMe-Val, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂.

4. (canceled)

5. The cyclic peptide of claim 1, wherein the first beta hairpin region comprises two contiguous residues independently selected from: L-Pro, D-Pro, L-Aze, D-Pip, and D-NMe-Val.

6. The cyclic peptide of claim 5, wherein for the two contiguous residues, one is D and the other is L.

7-8. (canceled)

9. The cyclic peptide of claim 6, wherein the two contiguous amino acid residues are D-Pro and L-NMe-Phe, wherein the phenyl group of L-NMe-Phe is optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂.

10-11. (canceled)

12. The cyclic peptide of claim 1, wherein the second beta hairpin region comprises a second two contiguous residues independently selected from: D-Pro, a peptoid, a D-N-alkylated amino acid, and an L-N-alkylated amino acid.

13-21. (canceled)

22. The cyclic peptide of claim 1, wherein the molecular weight of the cyclic peptide is from 800 to 1300 Da.

23-24. (canceled)

25. The cyclic peptide of claim 1, characterized by at least four amino acid residues comprising rings independently selected from optionally substituted monocyclic carbocycle and optionally substituted monocyclic heterocycle, wherein at least one of the monocyclic carbocycle and monocyclic heterocycle are substituted.

26. The cyclic peptide of claim 25, wherein the optionally substituted monocyclic carbocycle is phenyl and optionally substituted monocyclic heterocycle is a heteroaryl ring, wherein at least one phenyl or heteroaryl ring is substituted by one or more substituents independently selected from halo, —SCH₃, —SOCH₃, —SO₂CH₃, —OH, —CN, —NO₂, C₁₋₄ alkyl, —CH₃, —CF₃, —CHF₂, —OBz, —OCH₃, —OCF₃, —SF₅, and —OCHF₂.

27-29. (canceled)

30. The cyclic peptide of claim 1, characterized by at least four amino acid residues with side chains selected from -alkylene-(monocyclic carbocycle) and -alkylene-(monocyclic heterocycle), wherein the monocyclic carbocycle and monocyclic heterocycle are independently optionally substituted.

31-36. (canceled)

37. The cyclic peptide of claim 1, characterized by at least three amino acid residues comprising rings independently selected from optionally substituted phenyl and optionally substituted monocyclic heteroaryl, wherein each phenyl and heteroaryl ring is independently optionally substituted by one or more substituents independently selected from halo, —SCH₃, —SOCH₃, —SO₂CH₃, —OH, —CN, —NO₂, C₁₋₄alkyl, —CH₃, —CF₃, —CHF₂, —OBz, —OCH₃, —OCF₃, —SF₅, and —OCHF₂.

38-41. (canceled)

42. The cyclic peptide of claim 1, wherein at least three backbone nitrogen atoms of the cyclic peptide are tertiary nitrogens.

43. The cyclic peptide of claim 42, wherein four or five backbone nitrogen atoms of the cyclic peptide are tertiary nitrogens.

44-46. (canceled)

47. The cyclic peptide of claim 43, wherein one or more of the tertiary nitrogens have an optionally substituted C₁-C₆ alkyl substituent independently selected at each tertiary nitrogen and wherein substituents on C₁-C₆ alkyl are independently selected from halo, —SCH₃, —SOCH₃, —SO₂CH₃, —OH, —CN, —NO₂, C₁₋₄alkyl, —OBz, —OCH₃, —OCF₃, —SF₅, and —OCHF₂.

48-49. (canceled)

50. The cyclic peptide of claim 42, wherein each tertiary nitrogen is independently represented by: 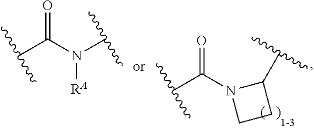
wherein R^{A} is C₁-C₆ alky optionally substituted with one or more substituents independently selected from halo, —SCH₃, —SO₂CH₃, —OH, —CN, —NO₂, C₁₋₄alkyl, —OBz, —OCH₃, —OCF₃ and —OCHF₂ and wherein 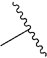
represents the point of connectivity to an adjacent amino acid residue.

51. (canceled)

52. The cyclic peptide of claim 1, wherein the cyclic peptide, has 10 amino acid residues.

53-99. (canceled)

100. The cyclic peptide of claim 1, wherein the cyclic peptide is selected from those in Table 3 and Table 4, or a pharmaceutically acceptable salt of any one thereof.

101. A pharmaceutical composition comprising a cyclic peptide of claim 1 and a pharmaceutically acceptable excipient.

102. A method of inhibiting MDM2, comprising administering a cyclic peptide of any one of claim 1.

103. (canceled)

104. A method of treating a disease or disorder in a subject in need thereof, comprising administering to said subject a therapeutically effective amount of a cyclic peptide of claim 1.

105. The method of claim 104, wherein the disease or disorder is cancer.

106. The method of claim 105, wherein the cancer is selected from acute lymphoblastic leukemia, acute myeloid leukemia, chronic lymphocytic leukemia, and chronic myeloid leukemia.

107. (canceled)

108. The method of claim 104, wherein the disease or disorder is associated with the proliferation of senescent cells, and wherein the disease or disorder associated with the proliferation of senescent cells is selected from a cardiovascular disease, an inflammatory disease, an auto-immune disease, a metabolic disease, a pulmonary disease, an ophthalmic disease, an otic disease, a renal disease, and a dermatological disease.

109. A method of inducing the death of a senescent cell in a subject in need thereof, comprising administering to said subject a therapeutically effective amount of a cyclic peptide of claim 1.
